(19) United States
(12) Patent Application Publication
(10) Pub. No.: US 2002/0187147 A1
(43) Pub. Date: Dec. 12, 2002

(54) Antigen specific recombinant MHC class II molecules and methods of use

(75) Inventors: Chih-Pin Liu, Cerritos, CA (US); Wei-Jen Lin, Cerritos, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 10/074,257

(22) Filed: Feb. 14, 2002

Related U.S. Application Data
(60) Provisional application No. 60/268,714, filed on Feb. 15, 2001.

(51) Int. Cl. G01N 33/53; G01N 33/567; C07H 21/04; A61K 39/395; C12P 21/02; C12N 5/06; C07K 14/74

(52) U.S. Cl. 424/144.1; 435/7.2; 435/69.1; 435/320.1; 435/325; 530/350; 536/23.5

(57) ABSTRACT

The present invention is directed to specific, multivalent, soluble recombinant I-Ag7/peptide complexes. These complexes are stable and are recognized by the TCR of T cells specific for a preselected antigen, to which they bind with low affinity. The inventive complexes may be used, for example, in the identification and study of autoreactive T cells and in the diagnosis, treatment and/or prevention of autoimmune or other diseases, including diabetes or pre-diabetic conditions.

This application claims priority from prior copending provisional patent application serial No. 60/268,714, filed Feb. 15, 2001.

This invention was made with government support in the form of grant numbers AI-44429 and AI-48847 from the National Institutes of Health (NIH)/The Juvenile Diabetes Foundation International (JDFI) and a Cancer Center Core Grant CA33752. The United States Government may have certain rights in the invention.

### BACKGROUND OF THE INVENTION

1. Technical Field

This invention relates to the field of recombinant nucleic acids, proteins, peptides, molecular complexes and methods useful for detection and modulation of specific immune receptors. In particular, the methods and materials are useful for detection of specific autoimmune T cells present in diabetes. Detection of GAD-specific T cells in diabetic and non-diabetic mice treated with GAD peptides revealed the induction of specific T cells that inhibit insulin-dependent diabetes mellitus.

2. Description of the Background Art

Type 1 diabetes (insulin-dependent diabetes mellitus, IDDM) is a T cell mediated autoimmune disease characterized by the destruction of insulin producing cells (β-cells) in the islets of the pancreas. In diabetic humans and in a recognized animal model that spontaneously develops IDDM, the non-obese diabetic (NOD) mouse, interactions among multiple alleles of disease-related genes are necessary to cause the destruction of β-cells. Genetic studies have shown that more than a dozen loci may be involved in the development of IDDM.

The major function of the MHC Class II proteins is to bind and present antigens to T cell receptors (TCRs). In the NOD mouse, the presence of the MHC Class II I-Ag7 complex results in greatly increased susceptibility to IDDM; diabetes spontaneously develops in NOD mice when I-Ag7 is present on both alleles. Transgenic expression of non-I-Ag7 Class II genes can protect these NOD mice from diabetes.

Susceptibility to IDDM is strongly influenced by MHC Class II genotype. The I-Ag7 complex in NOD mice is composed of a conserved I-Ad α chain paired with a unique I-Ag7 β chain that carries a substitution of His, Ser amino acid residues at positions 56, 57 in place of Pro, Asp. This linkage between amino acid substitutions in the β chains of I-Ag7 and diabetes applies to human Class II MHC molecules (such as DQ) as well, making the NOD mouse a well-accepted model for human IDDM and pre-IDDM states.

During the onset of diabetes, autoantigen specific diabetogenic T cells arise, which set a chain of events in motion that eventually results in β-cell destruction. Studies suggest that the appearance of these autoantigen specific T cells is dependent on alterations in the thymus selection process which result in the breakdown of self tolerance to immunodominant epitopes of self-antigens. Thus, in pre-diabetic and diabetic conditions, diabetogenic T cells expressing low affinity T cell receptors (TCRs) specific for diabetogenic autoantigens are not eliminated by negative selection in the thymus as they normally would be because they weakly bind to the altered MHC Class II molecule such as I-Ag7 or DQ, present in susceptible individuals. The unusual peptide interaction with the TCR present in diabetes when bound to I-Ag7 or DQ may be critical to the development of the disease. Analogous processes are believed to be responsible for the etiology of other autoimmune diseases, such as, for example, multiple sclerosis.

Several autoantigens that are presented by altered MHC complexes have been identified, including peptides derived from glutamic acid decarboxylase (GAD). Other autoantigens related to diabetes include, for example, insulin, IA-2 and heat shock proteins. Intrathymic, intravenous, or oral administration of GAD can delay the development of diabetes in NOD mice. Also, transfer of a T cell line reactive to the GAD peptide p524-543 transfers IDDM to recipient mice. Recent studies in transgenic mice provided direct evidence that expression of GAD is required for development of diabetes in NOD mice. Yoon et al., Science 284:1183-1187 (1999). Potential autoantigens for multiple sclerosis or the mouse model experimental autoimmune encephalomyelitis include, for example, myelin basic proteins (MBP), myelin oligodendrocyte glycoprotein (MOG) and proteolipid protein (PLP).

The detection and study of diabetic autoantigen specific T cells is made difficult by the low avidity with which I-Ag7 Class II molecules bind to peptide antigens. Antigen-MHC complexes are inherently very unstable and have been extremely difficult to produce and use in a stable form. I-Ag7 molecules present antigen only in the continued presence of the antigen; because avidity is so low, removing free peptide antigen from antigen-presenting cells in culture results in loss of peptide from the binding cleft. Kinetic studies of the binding of the MHC Class II-peptide complexes to TCR have showed the dissociation of the complexes with time. Savage et al., Immunity 10:485 (1999).

The weakness of the binding of antigen to I-Ag7 thus makes the use of MHC-antigen reagents for in vitro detection or other uses very difficult since the reagent will not bind T cells once peptide has been lost. The inherent spontaneous dissociation results in the rapid accumulation of highly unstable, empty MHC complexes which are not useful for T cell detection or treatment.

The use of labeled MHC-antigen complexes to detect specific classes of peptides has been discussed in the literature. Altman et al. (Science 274:94-96 (1996)) disclosed a bioengineered tetrameric peptide-MHC complex having a 15 amino acid peptide with a biotinylation site added to the carboxy terminus of HLA-A2 heavy chain and folded together with β₂-microglobulin and a specific peptide antigen. This complex, in which the peptide antigen was noncovalently bound to the ligand site, was then biotinylated and labeled with a phycoerythrin-avidin. This staining reagent was able to detect specific T cells when it was in multimeric form, presumably due to cooperative binding which increased the avidity for the complexes and the TCR. However, because this staining reagent involved noncovalent linkage of the antigenic peptide to the MHC heterodimer, the peptide could dissociate from the heterodimer. Savage et al., Immunity 19(4):485-492 (1999). Dissociation of the peptide from the intrinsically unstable I-Ag7 complex leads to complete dissociation of the I-Ag7 complex itself, resulting in a nonfunctional reagent.

Other authors have overcome the problem of poor binding by attaching the antigen peptide directly to the MHC molecule using methods such as photoaffinity labeling, which covalently attaches the peptide directly to the binding cleft of the MHC. See, for example, U.S. Pat. No. 5,734,023. These types of methods may result in the binding of a large proportion of the peptides in an incorrect orientation, or with incorrect stoichiometry. Thus, they produce reagents which cannot bind to TCRs predictably.

Crawford et al. (Immunity 8:675-682 (1998)) showed that linking the peptide to normal I-Ad MHC molecules using a flexible linker increased the stability of peptide binding. In the Crawford et al. study, the authors disclosed bioengineered I-Ad β chain molecules, bound via a flexible peptide linker to the antigenic peptide sequence. Some constructs included a peptide containing an Escherichia coli enzyme BirA biotinylation site. The Crawford et al. methods, however, do not produce tetramers which are suitable for in vivo applications, since the complexes could not be sufficiently purified.

Nag et al., U.S. Pat. No. 5,734,023, disclosed similar complexes. These authors indicated that complexes may be made directly from an encoding DNA using recombinant methods if the effector component of the complex is itself a protein. Complexes containing a peptide from the acetylcholine receptor (an epitope implicated in myasthenia gravis) linked to the N-terminal antigen binding site of an MHC polypeptide are discussed. As with the Crawford et al. methods, however, stable reagents pure enough for in vivo applications were not produced nor were any complexes suitable for detecting diabetic autoantigens, such as complexes comprising I-Ag7 or DQ MHC molecules.

Diabetic autoantigen-specific T cells (and autoantigen-specific T cells in general) are responsible for the proliferation of the immune response to the autoantigen which they display. The immune response results in inflammation and eventual destruction of β cells of the pancreatic islets and the development of frank symptoms of diabetes. However, some antigen-specific T cells have been difficult to detect and study due to their low numbers and to the generally low affinity with which they bind to their antigen. New methods to detect the presence of these antigen-specific T cells, particularly methods which are able to detect infrequent cells or cells which bind with low affinity, are needed. These methods would be able to detect a pre-diabetic state or other pre-autoimmune disease state before overt symptoms of the disease develop. Reagents which are able to detect members of specific T cell clones also may be used to provide methods of modulating the activity of the T cell clones, for example, inducing tolerance, expanding the clone or killing undesired T cells which bind the specific antigen. In addition, specific T cell clone identification can be useful for studying the effect of antigen treatment of IDDM by isolating the T cells clones produced in response to the treatment.

Since T cells reactive to GAD appear early in the development of IDDM, a method of reliable detection of small quantities of these cells would be advantageous in diagnosing prediabetic or diabetic conditions very early and providing a method of very early treatment or prevention of diabetic conditions. Such a method also would provide an invaluable tool in tracing the appearance of autoreactive T cells so that the early causes of IDDM can be studied.

Since many heterodimers associated with disease symptoms are intrinsically poor peptide binders and are relatively unstable compared to other MHC Class II molecules, it has been extremely difficult to generate soluble recombinant empty complexes stable enough to allow addition of peptides to the complexes. The previous methods available to create staining reagents and specific antigen-TCR binding complexes for diagnosis and treatment have not fully addressed this problem.

Soluble complexes of high stability, presenting specific peptide autoantigens previously have not been available to study the development and etiology of autoimmune diseases in mice and humans through detection of antigen-specific T cells. In addition, purified complexes in covalently linked multimeric form useful to modulate the expansion or activity of antigen specific T cells have not been available previously. Improved reagents and methods therefore are needed which can bind to and specifically recognize antigen specific T cells with greater sensitivity. Stable reagents and methods which can detect or modulate antigen-specific T cells are particularly needed in the art.

Recently, a selective approach for treatment of IDDM by immunomodulation has been developed. The method involves administrating specific protein or peptide antigens, such as GAD and its peptides, to animals. Tian et al., J. Exp. Med. 183:1561-1567 (1996); Tian et al., Nat. Med. 2:1348-1353 (1996); Cetkovic-Cvrlji et al., Diabetes 46:1975-1982 (1997); Tisch et al., J. Immunol. 163:1178-1187 (1999). This approach has been effective, however the mechanism underlying this effect of antigen-based immunotherapy remains elusive.

It has been hypothesized that antigen immunotherapy is epitope dependent and correlates with an induction of Th2 cells, but Th2 cells can also cause development of the disease. Pakala et al., J. Exp. Med. 186:299-306 (1997); Poulin and Haskins, J. Immunol. 164:3072-3078 (2000). Furthermore, a shift to Th2 cells after peptide treatment also may induce deleterious allergic or autoimmune responses. Kappos et al., Nat. Med. 1176-1182 (2000); Pedotti et al., Nat. Immunol. 2:216-222 (2001). A detailed understanding of the nature of the antigen-specific T cells that are induced by a particular antigen immunotherapy in treated animals will permit a better understanding of the immunoregulatory mechanisms of both successful and unsuccessful therapies, allowing workers to design more effective and less harmful immunotherapy treatments.

### SUMMARY OF THE INVENTION

Accordingly, this invention provides a recombinant nucleic acid which comprises DNA encoding an antigenic peptide sequence which binds to a Class II MHC molecule and DNA encoding the extracellular portion of the β chain of said Class II MHC molecule. In further embodiments, the recombinant nucleic acid further comprises DNA encoding the extracellular portion of the α chain of said Class II MHC molecule. Preferably, the α and β chains of said Class II MHC molecule lack a complete transmembrane region and the antigenic peptide sequence which specifically binds to a Class II MHC molecule is an autoantigen or a diabetic autoantigen, for example, a fragment of glutamic acid decarboxylase, such as the fragments provided in SEQ ID NOS:1-13 or fragments thereof. The recombinant nucleic acids of the invention may further comprise DNA encoding a biotinylation site, an oligohistidine sequence or both.

In further embodiments, the invention provides recombinant proteins which are encoded by the recombinant nucleic acids described above. Further, the invention provides heterodimers comprising recombinant proteins encoded by the recombinant nucleic acids described above. In yet further embodiments, the invention provides recombinant proteins described above which are biotinylated and which may further comprise a labeled avidin moiety non-covalently bound to the biotin.

In yet a further embodiment, the invention provides a stable molecular complex which comprises a recombinant protein which comprises a preselected peptidic antigen which binds to a Class II MHC molecule, the extracellular portion of a β chain of said Class II MHC molecule, and the extracellular portion of an α chain of said Class II MHC molecule. The invention provides stable molecular complexes which comprise any of the recombinant proteins described above.

In yet a further embodiment, the invention provides a method of detecting T cells which recognize a preselected peptidic antigen in a population of T cells which comprises providing a stable molecular complex such as those described above wherein the peptidic sequence is the preselected peptidic antigen, and wherein the stable molecular complex is labeled; incubating said stable molecular complex with the population of T cells under conditions such that the stable molecular complex binds to T cells in the population of T cells which recognize the preselected peptidic antigen; optionally removing unbound complexes; and detecting the labeled complexes on the T cells which recognize the preselected peptidic antigen.

In yet a further embodiment, the invention provides a method of diagnosing a diabetic or pre-diabetic in a mammal which comprises obtaining a sample from the mammal which contains a population of T cells; providing a stable molecular complex such as those described above, wherein the antigenic peptidic sequence is a diabetic autoantigen; incubating the stable molecular complex with the sample under conditions such that said stable molecular complex binds to T cells in said sample which recognize said diabetic autoantigen; optionally removing unbound complexes; and determining whether the stable molecular complex has bound to any T cells in the sample.

In yet a further embodiment, the invention provides a method of inducing tolerance to a preselected peptidic antigen in a population of T cells which comprises providing a stable molecular complex such as those described above and contacting the stable molecular complex with the T cells.

In yet a further embodiment, the invention provides a method of inducing or expanding protective clones of T cells which recognize a preselected antigen in a population of T cells which comprises providing a stable molecular complex such as those described above and contacting the stable molecular complex with the T cells.

In yet a further embodiment, the invention provides a method of killing T cells which recognize a preselected peptidic antigen in a population of T cells which comprises providing a stable molecular complex such as those described above, wherein the complex contains a toxin effector and contacting the stable molecular complex with the T cells.

In yet a further embodiment, the invention provides a method of inhibiting the onset of diabetes in a mammal in need thereof which comprises providing a stable molecular complex as described above wherein the antigenic peptide sequence is a diabetic autoantigen; contacting the stable molecular complex with a population of T cells allogeneic to the mammal under conditions such that the stable molecular complex binds to T cells in the population that recognize the diabetic autoantigen; separating from the T cell population T cells that bind to the stable molecular complex; and administering the separated T cells to the mammal.

### BRIEF DESCRIPTION OF THE FIGURES

FIG. 1 provides a map of the baculovirus transfer vector used to generate soluble recombinant I-Ag7/GAD protein complexes. TM=transmembrane region; Xa=Factor Xa recognition site; His=oligohistidine encoding sequence; BirA=BirA biotinylation site.

FIG. 2 shows data for IL-2 production by the indicated cell lines in response to I-Ag7 complexes.

FIG. 3 shows data for IL-2 production by the indicated cell lines in response to antigenic or irrelevant protein (left=response to p206; right=response to p524).

FIG. 4 provides histograms showing fluorescence activated cell sorting data of specific T cell hybridomas derived from NOD mice stained with tetI-Ag7/p206 complex in the absence (panels A and B) and presence (panels C and D) of H57 anti-TCR antibody.

FIG. 5 provides histograms showing fluorescence activated cell sorting data of specific T cell hybridomas derived from NOD mice, stained with tetI-Ag7/p524 complex in the absence (panels A and B) and presence (panels C and D) of H57 anti-TCR antibody.

FIG. 6A shows the fluorescence activated cell sorting results of NOD T cells from mice previously immunized with p206 peptide, stained with tetI-Ag7/p206 complex and anti-CD4 antibody.

FIG. 6B depicts the number of the CD4+ cells which stain with tetI-Ag7/p206 complex relative to CD4 staining.

FIG. 7A shows the fluorescence activated cell sorting results of NOD T cells from mice previously immunized with p524 peptide and stained with tetI-Ag7/p206 complex and anti-CD4 antibody.

FIG. 7B depicts the number of the CD4+ cells which stain with tetI-Ag7/p206 complex with relative to CD4 staining.

FIG. 8A shows the fluorescence activated cell sorting results of normal BALB/c splenic T cells from mice previously immunized with p206 peptide and stained with tetI-Ag7/p206 complex and anti-CD4 antibody.

FIG. 8B depicts the number of the CD4+ cells which stain with tetI-Ag7/p206 complex relative to CD4 staining.

FIG. 9A shows the fluorescence activated cell sorting results of NOD T cells from mice previously immunized with p206 peptide and stained with tetI-Ag7/p524 complex and anti-CD4 antibody.

FIG. 9B depicts the number of the CD4+cells which stain with tetI-Ag7/p524 complex relative to CD4 staining.

FIG. 10A shows the fluorescence activated cell sorting results of NOD T cells from mice previously immunized with p524 peptide and stained with tetI-Ag7/p524 complex and anti-CD4 antibody.

FIG. 10B depicts the number of CD4+cells which stain with tetI-Ag7/p524 complex relative to CD4 staining.

FIG. 11A shows the fluorescence activated cell sorting results of normal BALB/c splenic T cells from mice previously immunized with p524 peptide and stained with tetI-Ag7/p524 complex and anti-CD4 antibody.

FIG. 11B depicts the number of the CD4+cells which stain with tetI-Ag7/p524 complex relative to CD4 staining.

FIG. 12 shows staining results for CD4^{hi} and CD4^{low} cells by p206 specific complex.

FIG. 13 shows staining results for CD4^{hi} and CD4^{low} cells by p524 specific complex.

FIG. 14 shows the fluorescence activated cell sorting results of NOD T cells from mice previously immunized with p206 peptide and stained with tetI-Ag7/p206 complex and anti-CD4 antibody. The cells were uncultured (14A) or cultured with antigen (14B).

FIG. 15 shows the fluorescence activated cell sorting results of NOD T cells from mice previously immunized with p524 peptide and stained with tetI-Ag7/p524 complex and anti-CD4 antibody. The cells were uncultured (15A) or cultured with antigen (15B).

FIG. 16 provides data for the IL-2 production response of CD4⁺ T cells which are recognized by labeled I-Ag7/p206 tetrameric recombinant reagents (pos) or not recognized by the reagents (neg).

FIG. 17 shows fluorescence activated cell sorting results characterizing purified peptide-specific T cells from NOD (Figures A-D) or BALB/c (Figures E-H) mice by staining with I-Ag7 or I-Ad tetremer reagents and an anti-CD4 antibody.

FIG. 18 shows analysis of IL-2 production by NOD and BALB/c T cells staining positive or negative with the indicated tetramer reagent.

FIG. 19 shows results of ELISA analysis of IFNγ and IL-4 production by antigen-stimulated NOD or BALB/c mouse T cells which stain (+) or do not stain (−) with the indicated tetramer reagent.

FIG. 20 shows the IFNγ/IL-4 ratio for the cells indicated, treated as for FIG. 19.

FIG. 21 shows intracellular cytokine staining of cells staining with the indicated tetramer reagent.

FIG. 22 shows the percentage of mice remaining diabetes-free after adoptive transfer of NOD mouse splenocytes with either N206⁺, N221+ or both N206⁺ and N221⁺ T cells.

FIG. 23 shows analysis of IL-10 (Figures A and B) and TGFβ (Figures C and D) production by the indicated tetramer⁺ and tetramer⁻ cells after stimulation by the indicated antigenic peptide.

FIG. 24 shows results of co-staining N206⁺ and N221⁺ cells with the indicated reagents.

### DETAILED DESCRIPTION OF THE PREFERRED EMBODIMENT

The present invention provides reagents and methods for studying mechanisms underlying the development of autoreactive T cells, and for diagnosing and treating conditions in which specific T cell populations are an etiologic agent, including diabetic and pre-diabetic conditions related to autoreactive T cells. Purified soluble recombinant complexes covalently linked to and presenting antigenic peptides, such as autoreactive peptides and the like, are provided by this invention. These soluble recombinant peptide complexes are stable and additionally may be biotinylated so that functional moieties can be added via avidin-biotin binding. Class II MHC tetramers may be used to isolate CD4⁺ T cells from antigen treated diabetes-prone NOD mice and non-diabetes-prone BALB/c mice. The studies demonstrate that antigen treatment induced various diabetes-inhibiting Th2- and Tr1-like cells specific for two GAD peptides in NOD mice and different T cell responses to the same peptides in NOD and BALB/c mice.

Previous studies have shown that mutant Class II MHC molecules, such as I-Ag7 isolated from NOD mice cells, are unstable and very poor peptide binders. The present invention provides complexes in which the components are covalently linked, so that peptide-binding to the MHC binding cleft can occur with greater affinity, thus stabilizing the entire complex even in the absence of a carboxyl terminal leucine zipper, which has been reported to be required. Hausmann et al., J. Exp. Med. 189:1723 (1999). The stability of the recombinant MHC Class II peptide complexes of this invention is demonstrated by their ability to stimulate antigen specific T cells to secrete IL-2 in vitro. In addition, the complexes are able to detect antigen specific T cells present in very low numbers. This indicates that the antigenic peptides in the complexes are presented and bound to the MHC Class II antigen binding cleft properly and efficiently, since peptide/MHC Class II complexes are more stable when able to constitutively bind a peptide. Complexes made according to prior art methods have not been shown to exhibit such stability in solution. Peptides of different lengths were used to make MHC Class II complexes, showing that the peptide-binding groove of the complex is flexible enough to accommodate different length peptides and present them to TCR efficiently. Those of skill in the art will appreciate that any antigenic peptide able to bind to MHC Class II is suitable for use with this invention.

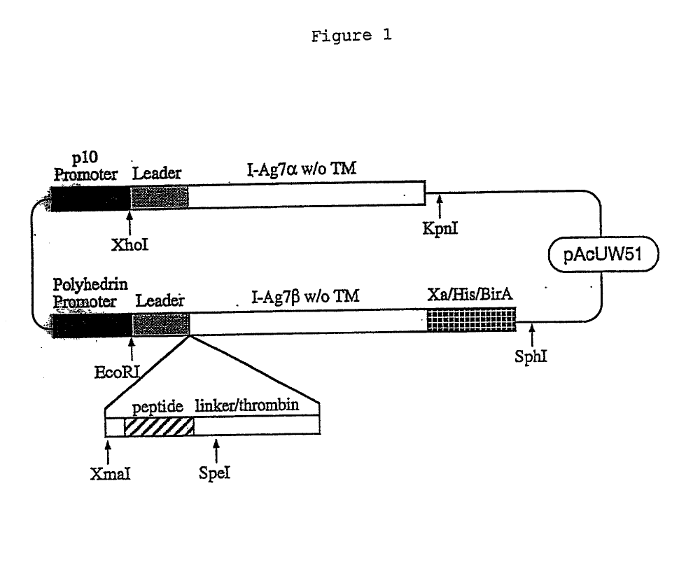

The complexes advantageously may be made using molecular biological techniques. Soluble MHC Class II molecules may be prepared by transducing cells, such as Hi5 and SF9 insect cells, using a baculovirus vector harboring the DNA construct shown in FIG. 1 or any convenient vector. Skilled artisans can easily adapt molecular biology techniques and methods to insert any desired DNA construct. A selected peptide (such as, for example, either GAD/p206 (TYEIAPVFVLLEYVT; SEQ ID NO: 1) or GAD/p524 (SRLSKVAPVIKARMMEYGTT; SEQ ID NO: 2) or any desired antigenic peptide) may be cloned to the 5′ end of an MHC β chain, preferably lacking the transmembrane and intracellular regions, so that the peptide is at the N-terminus of the expressed protein. The inventive method and complexes include those with I-Ag7 and DQ MHC molecules and are particularly suited to use for diabetic autoantigen T cells, but may be used with any MHC Class II molecule and any antigenic peptide which is recognized by the Class II MHC. At the 3′ end of the β chain, sequences encoding a series of histidine residues, such as an octomer of histidine residues, for purification purposes, and an Escherichia coli BirA biotinylation site may be added. In addition, a recognition sequence for Factor Xa may be added 5′ to both the His tag and the BirA site so that non-MHC peptide sequences (including the His tag and the BirA site) can be removed. See FIG. 1. Removal of non-relevant sequences is preferred for complexes which are to be used for in vivo applications. This can be accomplished by methods known in the art.

The use of a leader sequence is preferred, since a signal peptide assists in secretion of the recombinant protein into the culture medium. Preferably, the leader sequence is derived from an MHC gene, such as, for example, a mouse MHC gene. Exemplary sequences include the leader amino acid sequence MPCSRALILGVLALNTMLSLCGGEDD (SEQ ID NO:14) for the α chain and the leader amino acid sequence MALQIPSLLLSAAVVVLMVLSSPGTEG (SEQ ID NO:15) for the β chain.

Those of skill in the art are aware of methods of constructing functional vectors with variations to this described vector. Such variations are contemplated for use in the present invention, however the sequence of the antigenic peptide must be at the N-terminus of the recombinant protein (5′ to the MHC sequences).

The α and β chains of the MHC molecules preferably lack the transmembrane and cytoplasmic domains. This results in a shorter peptide which is easier to handle. Since the cytoplasmic region is not necessary for binding a peptide antigen, it may be omitted. Sequences which lack the transmembrane region are more easily soluble and will not spontaneously insert into membranes. Therefore, secreted proteins lacking a transmembrane region are preferred for ease of handling and purification.

Cells for expression of the recombinant protein (preferably eukaryotic, or most preferably insect or mammalian cells, or any convenient cell capable of expressing the desired protein) may be transfected according to known methods. Any method by which the DNA can be transferred conveniently is contemplated for use with this invention. The recombinant protein conveniently is expressed, secreted and purified from the cell media by known methods, such as affinity chromatography, gel filtration or any convenient method. Because both the α and β chains are present on the same two-promoter DNA vector, the two chains are produced at the same time and secreted into the culture medium. Thus, heterodimers may form in the culture medium spontaneously from the two expressed amino acid chains.

Many variations of these techniques are available in the art and are contemplated for use with this invention. For example, different cells may be used to express each of the MHC α and β chains. Preferably eukaryotic cells, most preferably mammalian or insect cells, or any cells capable of such expression may be used. The chains may be produced in different cells or the same cells using a single vector construct or more than one vector construct. Any vector system and methods compatible with the cells may be used to insert the recombinant DNA. Likewise, DNA molecules may be constructed by any method. Skilled artisans are well aware of these techniques for design, construction and expression of recombinant molecules and any of these methods are contemplated for use in the present invention. Preferably, however, the two chains are contained in a single construct expressed by a single cell type such that complexes can form in the medium bathing the cells after secretion.

Purification may be performed by any method known in the art. A skilled worker is able to design any suitable or convenient purification method compatible with the system of expression used and the structure of the recombinant molecule itself. The complexes may be used without further modification or may be modified to contain an effector molecule such as a label, toxin, cytokine or other molecule.

After purification, the MHC Class II/peptide complexes may be biotinylated and incubated with an avidin-effector molecule such as streptavidin-PE (SAPE) to generate multivalent complexes containing a label or other effector molecule. The recombinant protein complexes are conveniently biotinylated using the E. coli enzyme BirA, however, any method is contemplated for use by this invention. Those skilled in the arts of molecular biology and biochemistry are capable of designing different methods to accomplish biotinylation of a recombinant protein and these methods are contemplated for use with the invention.

After biotinylation, recombinant proteins may be incubated with streptavidin which has been labeled with phycoerythrin or any other suitable fluorescent molecule, or with any other label, such as colloidal gold, an enzyme (for example, alkaline phosphatase or a peroxidase) and the like. Labels or other effector molecules may be conveniently attached or non-covalently bound by other means, and any convenient method known in the art may be used in accordance with the present invention. Other methods of labeling the heterodimer may also be used, however biotinylation followed by addition of labeled avidin is preferred for many in vitro uses. Toxin effector molecules may also be used to specifically kill T cells either in vitro or in vivo.

Alternatively, the recombinant complex may lack a biotinylation site, or any other label. In such cases, a labeled antibody may be used to detect the complex, if desired. Antibody detection may be used with the inventive complexes in a diagnostic protocol, or as an adjunct to purification, or whenever detection is desired. Those of skill in the art are well aware of numerous techniques to make polyclonal or monoclonal antibodies to a desired protein or peptide and can easily devise an antibody detection method suitable for use with the various embodiments of this invention, including labeling of the antibody directly, or the use of labeled secondary antibodies. Because biotin-avidin systems generally are not suitable for use in vivo, non-biotinylated embodiments are preferred for such uses.

Avidin also may be coupled to a toxin or other effector molecule or to both a label effector molecule and a second effector molecule such as a toxin. For example, I-Ag7/peptide complexes coupled to a toxin may be used to kill autoreactive T cells which recognize the peptide in context with I-Ag7. Toxins or other effector molecules alternatively may be coupled to the recombinant protein by any desired method, such as for example, molecular cloning and the like. Therefore, the inventive reagents may be used in one embodiment for treatment of IDDM or for prevention of IDDM symptoms and β cell death, as well as diagnosis of IDDM and identification of autoreactive T cells. The term “effector,” therefore, signifies any molecule which can affect a T cell to which the complex may bind, including labels, toxins, stimulatory cytokines, and the like.

The peptide which is cloned to the MHC Class II β chain may be any antigenic peptide. Preferred antigenic peptides are those which are suspected in the etiology of autoimmune diseases such as multiple sclerosis or IDDM. Any β cell surface antigen which could be involved in the T cell mediated destruction of islet β cells also is contemplated for use with the invention. Peptides of various lengths are suitable, however preferred peptide sequences are about 10 to about 20 amino acids, or most preferably about 10 to about 12 amino acids. Any antigenic peptide or autoantigenic peptide, of any length, which may be presented in the context of MHC Class II, and particularly in the context of I-Ag7, DQ or any MHC molecule associated with a disease state, is suitable for use with the invention. In addition, immunologically equivalent peptide variants and fragments or synthetic peptides may be used, so long as the peptide sequence results in specific recognition of the desired population of T cells. “Immunologically equivalent” variants and fragments indicate those sequences which are recognized by the desired T cells.

Peptides derived from proteins which play a role in T cell mediated immune destruction or other T cell mediated conditions are particularly useful. These proteins include, but are not limited to, insulin, glutamine decarboxylase (GAD), IA-2, heat shock protein, myelin basic proteins (MBP), myelin oligodendrocyte glycoprotein (MOG) and proteolipid protein (PLP). Peptide sequences derived from these proteins which are recognized by autoreactive T cells and specifically by autoreactive diabetic T cells are particularly useful. Treatment of NOD mice with GAD protein or peptides reduced the incidence of diabetes. Tian et al., J. Exp. Med. 183:1561-1567, 1996; Tian et al., Nat. Med. 2:1348-1353, 1996; Cetkovic-Cvrlji et al., Diabetes 46:1975-1982, 1997; Tisch et al., J. Immunol. 163:1178-1187, 1999. In addition, a majority of T cell hybridomas derived from GAD protein treated NOD mice reacted with GAD p206 and p221 peptides, suggesting that these are immunodominant T cell epitopes. Chao and McDevitt, Immunogenetics 46:29-34, 1997; Chao et al., Proc. Natl. Acad. Sci. USA 96:9299-9304, 1999. Preferred peptides therefore include GAD/p206 (SEQ ID NO: 1), GAD/p524 (SEQ ID NO: 2) or other GAD peptides (see Table I, below) and immunologically equivalent variants or fragments thereof.

TABLE I
Exemplary Human and Murine Diabetic Autoantigens

| Peptide | | SEQ | |
|---|---|---|---|
| Name | Species | ID NO. | Sequence |
| GAD65 | human, | 1 | TYEIAPVFVLLEYVT |
| p206-220 | murine | | |
| GA065 | human, | 2 | SRLSKVAPVIKARMMEYGTT |
| p524-543 | murine | | |
| GAD65 | human, | 3 | LKKMREIIGWPGGSG |
| p221-235 | murine | | |
| GAD65 | murine | 4 | NMYAMLIARYKMFPEVKEKG |
| p247-266 | | | |
| GAD65 | human | 5 | NMYAMMIARFKMFPEVKEKG |
| p247-266 | | | |
| GAD65 | human, | 6 | KKGAAALGIGTDSVI |
| p286-300 | murine | | |
| GAD65 | human, | 7 | LVSATAGTTVYGAFD |
| p331-345 | murine | | |
| GAD65 | human, | 8 | PLQCSALLVREEGLM |
| p401-415 | murine | | |
| GAD65 | human, | 9 | WLMWRAKGTTGFEAH |
| p456-470 | murine | | |
| GAD65 | murine | 10 | VPPSLRTLEDNEERMSRLSK |
| p509-528 | | | |
| GAD65 | human | 11 | IPPSLRTLEDNEERMSRLSK |
| p509-529 | | | |
| GAD65 | human, | 12 | GDKVNFFRMVISNPA |
| p551-565 | murine | | |
| GAD65 | human, | 13 | ISNPAATHQDIDFLI |
| p561-575 | murine | | |

The methods and reagents of this invention may be used as a tool in the laboratory to study the trafficking of specific T cells, to monitor their numbers, to detect specific T cell populations in a sample or population of T cells, to isolate specific T cells, to induce tolerance to an antigen or to modulate the function of specific T cell populations (clones) in vitro and in vivo, including expanding the clones or killing them.

In vitro diagnostic tests for autoimmune disease, even in very early stages before overt symptoms are observed, may be performed according to the present invention. To perform such tests, a sample of cells containing T cells is obtained from a mammal. Samples for testing may be any convenient sample containing T cells and may be taken from any tissue having such cells. Splenic tissue is convenient, however thymus, lymph nodes or peripheral blood also may be used or any tissue in which T cells may be found. Organs containing infiltrating immune cells also may be used, or biopsy samples from such organs or tissues. Purified populations of T cells are most preferably used. Semi-purified T cell samples also are useful. If desired, T cell samples may be purified (enriched for T cells) using any suitable technique, for example an antibody affinity column, lymphocyte separation medium or nylon wool.

MHC Class II molecular complexes having covalently linked peptides according to the invention are then incubated with the sample T cells under conditions such that the complexes are able to bind to T cell receptors on cells reactive to that particular peptide. More than one complex may be incubated with the T cells either together or separately to detect multiple T cell groups simultaneously or sequentially. In such tests, each complex preferably has a different label to differentiate between the different complexes and the different populations of specific T cells. Unlabeled complexes may be used in diagnostic assays as well. These unlabeled complexes may be detected using labeled antibodies or any convenient method.

Generally, suitable conditions for incubation include physiological or neutral pH (such as pH 7.0-7.4) in an appropriate buffer or medium, such as phosphate-buffered saline, RPMI 1640 or the like, at a temperature of about 4° C. to about 37° C. Incubation times are desirably from about 1 hour to about 4 hours, or any convenient time. The most preferred conditions for incubation include the use of a cell culture medium such as RPMI 1640 with 5% fetal calf serum (FCS), pH 7.0, 37° C., and an incubation time of about 3 hours.

When detecting specific T cell populations in an in vitro assay, the staining intensity may be improved by enhancing the binding affinity of the complexes to antigen specific TCRs. Adding an antibody which binds to the constant region of a TCR, such as the pan-TCR Cb antibody, H57-597 (H57) to the incubation mixture accomplishes this. Any antibody or other substance which is able to cluster TCRs also may be used for this purpose. Because H57 antibody binds to the constant region of the beta chain of TCRs, it is able to enhance staining by cross-linking the TCRs expressed on the cell surface. This increases the binding of I-Ag7-peptide complexes to the clustered TCRs. A concentration of approximately 1 μg H57 per 10⁶ cells added together with the I-Ag7 tetramers is convenient, but it is contemplated that this concentration may be adjusted by a skilled artisan to suit the needs of the assay.

Of course, those of skill in the art are capable of modifying the assay conditions to optimize performance for their own particular reagents and this is contemplated by the invention. Therefore, conditions falling outside the ranges discussed above are contemplated for use with the inventive methods. Incubations preferably are performed with the complex present at a concentration of about 10 μg/ml to about 100 μg/ml. The complex is most preferably at a concentration of about 30 μg/ml to about 60 μg/ml. However, higher or lower concentrations may be suitable for some methods, depending on the other conditions used and or the binding strength of the T cells to the particular peptide or peptide complex used. Variation and modification is therefore contemplated for use with this invention.

After incubation, a wash step may be used to remove unbound complexes. If unlabeled complexes are used in the method, bound complexes may be detected on the T cells by any convenient method, such as direct or indirect antibody labeling. If labeled complexes are used, the labeled T cells may be detected by any convenient method. For methods involving both labeled complexes and alternate labeling methods, the method of detection is chosen depending on the label chosen. For example, if a fluorescent label or labels are chosen, the T cells binding the complex may be detected by methods such as fluorescence-activated cell sorting or various histochemical techniques. Those of skill in the art are well acquainted with different methods of labeling and detection, and therefore may select any suitable method which is compatible with the cell and labels being detected. For example, methods involving enzymes would require detection methods including substrates for the enzyme chosen.

In methods involving detection of specific T cell clones, the sample or population of T cells being assayed may be stimulated in vitro prior to incubation with the inventive complexes. Such stimulation may be performed by incubating the population of T cells with the peptide antigen for which the T cells to be detected are specific. Such incubations may be performed using the naked peptide antigen, a modified or variant peptide having the same T cell binding properties, or using allogeneic antigen presenting cells, which present such a peptide antigen, modified peptide antigen or variant peptide antigen. In vitro stimulation in this manner increases the number of the specific T cells in the T cell population, making binding and detection by the complexes easier and increasing the binding signal for T cell clones present in very small number.

The general considerations and specific methods discussed above in the context of a diagnostic assay may be applied to laboratory studies on specific T cell trafficking by assaying different tissues for the presence of specific T cells or assaying tissues at different times. Likewise, the same methods may be used to monitor the number of specific T cells in a patient or in a sample. In addition, specific T cell populations may be isolated using the inventive peptide complexes by any separation method known in the art. For example, fluorescent labeled complexes may be used to stain cells which may be separated by fluorescence-activated cell sorting. Alternate methods such as magnetic bead separation techniques, microdissection techniques, various antibody-directed methods and the like, or any convenient method also may be used.

The peptide complexes of this invention also may be used for treatment of patients or any mammal suffering from a diabetic or prediabetic condition or any other condition where the presence of specific T cell clones is a causative factor, such as multiple sclerosis, experimental autoimmune encephalomyelitis or other autoimmune diseases. Peptidic complexes which recognize and bind to destructive T cells may be administered to a patient. Complexes bearing a toxin molecule may be used to target the toxin to those specific destructive T cells, such as diabetic or multiple sclerosis autoantigen responsive T cells or any specific T cell population, to specifically kill the T cell population responsible for a particular disease condition. Peptide complexes without a toxin may also be used to induce death of specific T cells. Complexes having no effector can bind to the specific T cells which recognize the bound peptide in the complex inducing them to change their responsive kinetics, resulting in temporary activation, followed by cell death.

Treatment of patients by activation or expansion of specific, beneficial T cell clones also may be performed in the same manner, using peptide complexes having no effector or containing an effector such as a stimulatory cytokine, for example IL-4, IL-10 and the like. In addition, the inventive peptide complexes may be used to induce tolerance in a patient suffering from an overabundance of T cell reactivity to a particular peptide antigen. Such methods may be used to treat allergies by inducing tolerance to the antigen which causes the allergic reaction. The peptide complexes also may be used to vaccinate a patient by inducing expansion of specific T cell clones which protect a patient upon later exposure to that antigen.

In addition, the methods disclosed here may be used in conjunction with various peptide vaccine therapies. Where it is desirable to use a peptide vaccine, the inventive methods advantageously may be used to study, detect or quantitate specific T cells responsive to the peptide antigen(s) used in the vaccine. The methods may be used to study a patient's response to the specific vaccine antigen(s) before vaccine administration and to gauge the patient's response to the vaccine post-administration. The methods, therefore, may be extremely helpful in determining the effectiveness of particular vaccines in a clinical or clinical trial scenario. The complexes of this invention also may be used in conjunction with traditional peptide vaccine therapies to expand or treat specific T cell population(s) which respond to the vaccine antigen.

Therapeutic compositions, including vaccines, containing the inventive antigen-specific T cell binding complexes will contain a therapeutically effective dose of the complexes. The dose, carrier and route of administration selected will depend upon, among other factors, the condition of the patient, the desired route of administration, and the activity of the complex with respect to the specific T cell to which it binds. This is readily determined and monitored by the physician during the course of therapy.

One carrier for infusion or injection of the complexes is a sterile isotonic aqueous solution, for example, normal saline or 5% dextrose, optionally containing buffers or other physiologically compatible excipients. Alternatively, the complexes may be packaged in liposomes. Therapeutic compositions may be administered by oral, subcutaneous, intramuscular, intravenous, intraperitoneal, intranasal, transmucosal, or other conventional routes or by inhalation. The skilled artisan is fully capable of compounding the complexes into formulations which are suitable for administration of therapeutically effective doses of complexes by the above-mentioned routes, or by any other route, such as transdermal, including inhaled forms of the complexes or direct injection into the site of T cell directed damage. Any of the pharmaceutical compositions may optionally be administered in conjunction with immune adjuvants, if desired.

Dosage amounts and intervals may be adjusted individually to provide plasma levels of the complex which are sufficient to maintain therapeutic effect. Usual patient doses for systemic administration range from about 1-500 μg/kg, commonly from about 10-100 μg/kg, and most preferably from about 20-50 μg/kg. In cases of local or selective administration, the effective local concentration may not be related to plasma concentration. One having skill in the art will be able to optimize therapeutically effective local dosages without undue experimentation. The amount of complex administered will, of course, be dependent on the subject being treated, the subject's weight and overall physical condition, the severity of the affunction being treated, the manner of administration and the judgement of the prescribing physician.

Therapeutic efficacy of the active complexes can be determined by standard pharmaceutical procedures in cell cultures or experimental animals, e.g., by determining the LD₅₀ (the dose lethal to 50% of the population), and the ED₅₀ (the dose therapeutically effective in 50% of the population). The dose ratio between toxic and therapeutic effect is the therapeutic index. The data obtained in these culture assays and animal studies can be used in formulating a dosage range that is effective but not toxic in humans.

In the examples provided below, antigen-based immunotherapy induced T cells with cytokine secretion profiles which varied depending on the antigen specificity and the strain of mice from which the T cells were derived. N206+ and N221+ T cells secreted different amounts of IFNγ but comparable amounts if IL-2, IL-4 and IL-10. Despite this difference, both T cells were able to inhibit IDDM. One possibility for this inhibitory effect was that both of these cells contained IL-4/IL-10 co-producing Th2-like cells and IFNγ/IL-10 co-producing Tr1-like cells. These cells produced a large amount of immunosuppressive IL-10 or IL-4. It is likely that both cell populations negatively regulate a Th1 response and result in IDDM inhibition. Interestingly, although both N206+ and N221+ cells produced comparable amounts of IL-4 and IL-10, N206+ cells had a stronger inhibitory effect on IDDM. This difference could be due to the finding that N221+ cells produced 8- to 10-fold more IFNγ than did N206+ cells. This was probably because N221+ cells contained 25-fold more Tr1 cells and some Th1 cells. While the effect of higher levels of IFNγ secreted by N221+ cells on IDDM is not clear, it could serve to counteract the inhibitory effect if IL-4 or IL-10. Therefore, Tr1 cells could inhibit not only Th1 but also Th2 responses. Additionally, N221+ but not N206+ cells contained some Th1 cells which may also counteract the effect of Th2 cells.

Interestingly, triple transfer resulted in an inhibitory effect on IDDM which was similar to that seen in the double transfer fo N206+ but not N221+ cells. See Example 13. This observation may be due to the fact that a smaller number of N221+ cells producing high levels of IFNγ were used in the triple transfer studies, with resultant lower levels of IFNγ.

The inhibitory effect of tetramer+ T cells could be due to differential expansion or homeostatic competition of tetramer+ T cells vs. diabetogenic splenocytes in animals as described in Theofilopoulous et al., J. Clin. Invest. 108:335-340, 2001; Salomon et al., Annu. Rev. Immunol. 19:225-252, 2001. The tetramer+ T cells hay have a growth advantage over the diabetogenic splenocytes. The low numbers of N206+ or N221+ cells (2 to 4%) detected in the spleens of the co-transferred animals suggest that they proliferated slowly but were sufficient to inhibit diabetes.

Surprisingly, T cells from similarly treated BALB/c mice were essentially nonpolarized or slightly biased toward Th1 cells. Therefore, the atypical cytokine secretion profile of N221+ and N206+ cells is likely intrinsic in nature in NOD mice but not in BALB/c mice. One might have predicted that BALB/c mouse T cells were more likely to become Th2 cells due to their genetic backgrounds. However, in addition to being less polarized in producing IFNγ/IL-4, the B206+/B21+ cells secreted more IL-2 but less IL-4/IL-10 than did N206+/N221+ cells. It is likely that these differences are due to the unique properties of I-Ag7 in binding and presenting peptides or the nature of the autoantigen/MHC complex. While the weaker IL-2 response can be interpreted as an indication that N206+/N221+ cells bore TCRs of lower affinity than B206+/B221+ cells for their ligands, it is not clear whether the same rationale can be applied to explain the differences in IL-4/IL-10 production. Alternatively, the difference could be due to altered APCs in NOD mice. Altogether, the results show that treatment of NOD mice but not BALB/c mice induce polarized population of T cells that include regulatory cells characteristic of either Th2 or Tr1 cells. The studies also indicate that regulatory T cells secreting lower amounts of IFNγ have a stronger regulatory effect on IDDM than those secreting higher levels of IFNγ.

The following non-limiting examples are provided.

### EXAMPLES

### Example 1

### Construction, Expression and Purification of a GAD Peptide Specific I-Ag7 Complex

Soluble I-Ag7 molecules with covalently linked peptides were prepared by infecting insect cells with baculovirus vectors containing DNA constructs described below according to prior art methods. DNA constructs for insertion into baculoviruses vectors were generated by cloning sequences of a selected peptide (e.g., GAD/p260 or GAD/p524) to the 5′ end of the I-Ag7 β chain which lacked the transmembrane and intracellular region and the I-Ag7 α chain also lacking the transmembrane and intracellular region. See FIG. 1.

The I-Ag7 γ chain also contained in its 3′ end sequences encoding a Factor Xa site, a peptide of 8 Histidine residues for purification purposes, and a peptide capable of being biotinylated. The sequences of the DNA constructs inserted into the vector at the ₅′ end of the truncated I-Ag7 β chain are provided as SEQ ID NOS:16 and 17, below:

| | |
|---|---|
| 5′-cccgggactgagggcacctatgagatcgcccctgtatttgtgctgctagaat | (SEQ ID NO:16; GAD206) |
| atgttacaggaggtgggggctcactagt-3′ | |
| 5′-cccgggactgagggcagccgcctctcaaaggtggcgccagtgattaaagcca | |
| gaatgatggagtatgggaccacaggaggtgggggctcactagt-3′ | (SEQ ID NO:17; GAD524). |

The sequence of the DNA construct inserted into the vector encoding the Xa, His8, and biotinylation peptide is provided as SEQ ID NO:18, below:

5′-atcgagggacgtggaggtcatcatcatcatcatcatcatcatgctagcgg
cggtggacttaacgacatctttgaggcacagaagatcgagtggcacgagtgagcatgcgg
atcc-3′ (SEQ ID NO:18).

The mouse MHC leader sequences SEQ ID NOS:14 and 15 were used for the α and β chains, respectively. The basic protocol involved cloning of the relevant genes into transfer plasmid vectors under the control of either the baculovirus polyhedrin promoter or the p10 late promoter. Standard cloning methods for construction of the expression vectors are described in Molecular Cloning—A Laboratory Manual, Cold Spring Harbor Laboratory, Cold Spring Harbor, N.Y. (1989).

Cells were infected according to the manufacturer's instructions (SF9 and Hi5 cells, PharMingen, San Diego, Calif.). Briefly, insect cells at a density of 2×10⁶ cells/ml were infected with recombinant virus at an MOI of S to 10 in a spinner flask. The cell culture supernatant usually was harvested about 3-5 days post infection. The expression vector DNA plasmid was co-transfected with baculovirus DNA into SF9 insect cells. Rescued recombinant lytic virus was recovered from the culture supernatant and a subsequent infection was used to produce a high titered viral stock. This stock was used to infect SF9 or Hi5 insect cells at high multiplicity to produce soluble protein. The protein was produced late in the infection (about day 3 to day 5) and was recovered from the culture supernatant.

The soluble recombinant I-Ag7/p206 and I-Ag7/p524 protein complexes were purified from the culture supernatant using Ni beads (Qiagen, Valencia, Calif.) added to the culture supernatant or a Ni bead affinity column, and further purified through a Superdex 200 HR 10/30 gel filtration column (Pharmacia, Piscataway, New Jersey) with PBS at a flow rate of 0.5 ml/min and detection at OD 280 nM. The soluble recombinant I-Ag7/p206 and I-Ag7/p524 protein complexes had apparent molecular weights of 65-67 kD as heterodimers and 33-35 kD as individual α and β chains as detected on a 10-15% gradient SDS-PAGE PhastGel with a PhastSystem separation and control unit apparatus (Pharmacia, Piscataway, N.J.). The purified I-Ag7/peptide heterodimers were then biotinylated (see Example 2).

### Example 2

### Biotinylation and Fluorescent Labeling of I-Ag7/GAD-Peptide Complexes

Purified I-Ag7/peptide complexes were biotinylated using a kit containing the enzyme BirA according to the manufacturer's instructions (Avidity, Denver, CO). The biotinylated protein was further purified by another gel filtration step using the column described in Example 1, in phosphate buffered saline at pH 7.0 and a flow rate of 0.5-1.0 ml/min. The protein peak containing the α/β heterodimer was collected. Biotinylated proteins were incubated with streptavidin-phycoerythrin (SAPE) at a calculated molar ratio of 4 to 1 (4 molar of I-Ag7/peptide complex to 1 molar of SAPE) overnight at room temperature. Labeled multivalent complexes were purified by FPLC chromatography using a Superdex G200 sizing column (Pharmacia, Piscataway, N.J.).

### Example 3

### Surface Staining and Flow Cytometry Analysis

Surface staining of T cells with the fluorescent-labeled complexes may be performed by adopting prior art methods. The complexes were incubated with the cells at 37° C. for 2-3 hours in RPMI medium containing 10% FCS in the presence of blocking agents including normal mouse serum and cell culture supernatant containing the anti-Fc receptor antibody 2.4G2. The final concentration of the complexes used in the staining reactions was 30 μg/ml. For each staining experiment, at least 5×10⁵ cells were analyzed on a FACSCaliber Instrument (Beckton Dickinson, Inc., Mountain View, Calif.).

### Example 4

### Generation of Antigen Specific T Cell Hybridomas

To demonstrate the method of the invention, NOD mice and BALB/c mice were immunized and used to generate antigen specific T cell hybridomas. Both strains of these mice were purchased from The Jackson Laboratory and housed and bred in a specific pathogen free environment at the City of Hope animal facility. Female NOD mice developed diabetes as early as at 12 weeks of age. More than 60% of the female mice became diabetic by the age of 20 weeks old. Mice were used at 8 to 12 weeks old (pre-diabetic) in the studies, unless otherwise stated.

NOD T cell hybridomas were produced according to prior art methods. Briefly, NOD mice were immunized subcutaneously at the base of the tail with 100 μg GAD peptide p206 (SEQ ID NO: 1) or p524 (SEQ ID NO: 2) in complete Freund's adjuvant. Draining lymph nodes were removed seven days later and cultured with antigen in Click's medium for four days and then in IL-2 medium for three days. Antigen specific blasts were selected using lymphocyte separation medium. The above blasts were fused with BW5147α⁻β⁻ cells, a thymoma cell line deficient in the expression of TCR α and β chains described in White et al., J. Immunol. 143-1822 (1989).

These antigen specific hybridoma cells were screened based on their ability to secrete IL-2 in response to the antigenic peptide by known methods. Briefly, for stimulation of cells using peptides at different concentrations, 50,000 hybridoma cells were cultured with NOD spleen cells as the antigen presenting cells. After 24 hours in culture, production of IL-2 was determined using an IL-2-dependent indicator cell line (HT-2). HT-2 cells do not survive in the absence of IL-2, therefore the presence of IL-2 in the culture supernatant of antigen specific T cells secreted in response to activation by antigen allows the cells to survive.

For each of the two GAD peptides, more than seventy-two different hybridoma cells were screened and tested for IL-2 secretion in response to their respective antigenic peptides presented by NOD mouse spleen cells. Hybridomas (5-10×10⁴ cells/well) were incubated with GAD/p206 or GAD/524 in RPMI 1640 containing 5% FCS, pH 7.0 at 37° C. for 3 hours. Resulting IL-2 secretion was measured in response to stimulation with antigenic peptide at concentrations of 0-50 μg/ml for screening assays and of 12.5-25 μg/ml for stimulation assays of known responders. Any suitable concentration may be used, depending on the particular goals of the assay. IL-2 secretion was determined using 3-[4,5-dimethylthiazol-2-yl]-2,5-diphenyl-tetrazolium bromide (MTT) incorporation by the live cells, measured using an ELISA reader.

The GAD/p206 and GAD/p524 specific hybridoma cells, hereinafter NOD206 and NOD524 cells respectively, were tested for the ability to specifically recognize the immunizing peptide without cross-reactive recognition of the other peptide. For each of the peptides p206 and p524, twenty-five hybridoma cells were tested for their response to their antigenic peptide in order to identify the hybridoma cells with higher sensitivity to antigen stimulation. Two representative hybridomas reactive to GAD/p206 (NOD206-37 and NOD206-103) and GAD/p524 (NOD524-5 and NOD524-20) were tested for IL-2 secretion in response to their specific antigen and for TCR expression level. The NOD206-103 and NOD524-20 hybridomas demonstrated a higher affinity to their respective ligands than did the NOD206-37 and NOD524-5 hybridomas. The data also showed that these hybridomas could be stimulated only by their antigenic peptide, but not by the other irrelevant peptide.

The TCR expression level on these four hybridomas were compared by staining with the pan-TCR Cb antibody, H57-597 (H57). The results showed that the NOD206-37 cells expressed TCRs at a level slightly higher than that of NOD206-103 cells and that the NOD524-5 cells expressed TCRs at a level slightly higher than that of NOD524-20 cells. Thus, the difference in the sensitivity in antigen responses above was not due solely to differences in TCR densities on the cell surface. The combined results of the antigen stimulation and TCR density experiments demonstrate that the TCRs expressed on the NOD206-103 cells have a greater affinity to the I-Ag7/p206 ligand than do the TCRs expressed on NOD206-37 cells. Similarly, the TCRs expressed on the NOD524-20 cells have a greater affinity to the I-Ag7/p524 ligand than the NOD524-5 cells.

The two GAD self-peptide specific complexes, tetI-Ag7/p206 and tetI-Ag7/p524, stained I-Ag7 selected T cells only weakly. It has been shown that the level of binding or the staining intensity of tetramers to TCRs correlates with the affinity of TCRs for MHC/peptide ligands. Therefore, these results indicate that the TCRs have very low binding affinity to the recombinant I-Ag7/GAD peptide complex ligands. The multivalent I-Ag7/peptide complexes were able to detect antigen specific T cell hybridomas and T cells derived from NOD mice, however. This indicates that GAD peptide-specific low-affinity TCRs were selected by and detected by the inventive I-Ag7 complexes. Immunization of NOD mice increased the selection/expansion of potentially higher affinity TCRs which could be detected more easily by tetramers. Analysis of T cells from NOD mice of different ages showed that the T cells reactive to the two GAD peptide self-antigens were present in very low frequency in NOD mice spleen. Therefore, the diabetes-associated I-Ag7 molecules selected for very low frequency self-antigen reactive low affinity T cells. The inventive method therefore was able to detect the specific population of autoreactive T cells which are of interest, in spite of their low number and weak binding.

### Example 5

### Analysis of IL-2 Production by Antigenic Specific T Cell Hybridomas

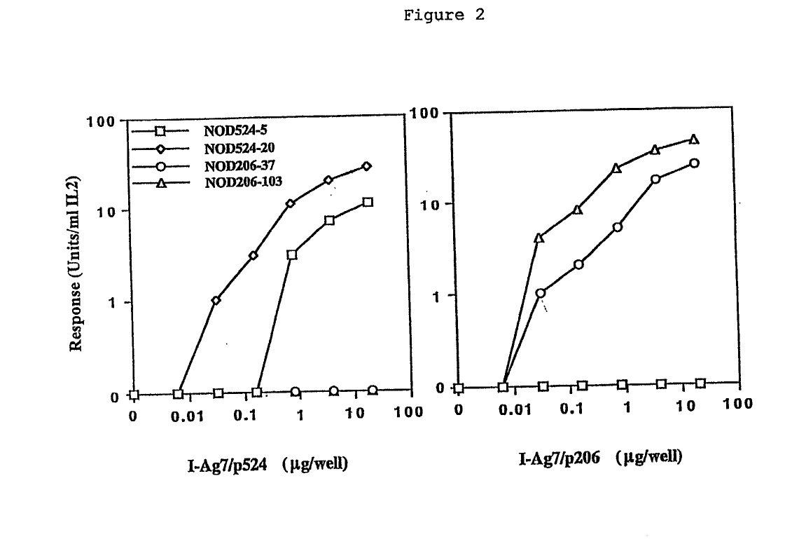

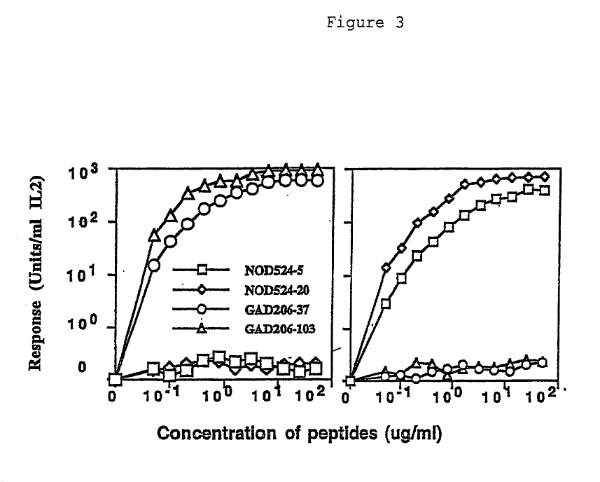

NOD mouse T cell hybridomas were stimulated with various concentrations of plate-bound recombinant I-Ag7/p206 and I-Ag7/p524 proteins. The proteins were diluted with a 5-fold serial dilution starting with 20 μg/ml and allowed to bind to the plate at 4° C. overnight. Two representative hybridoma cells for each protein (NOD206-37 and NOD206-103 cells for I-Ag7/p206; NOD524-5 and NOD524-20 cells for I-Ag7/p524) were incubated with protein at 5×10⁴ cells/well for 24 hours. IL-2 production was measured and is reported in FIG. 2. This experiment was repeated, performing an incubation for each cell type with its antigenic protein and with the irrelevant protein. See FIG. 3. The hybridomas were stimulated only by their antigenic peptide but not the other irrelevant peptide, showing that the IL-2 production responses are antigen specific.

### Example 6

### Detection of T cell Receptors Expressed on I-Ag7 Selected Hybridoma Cells

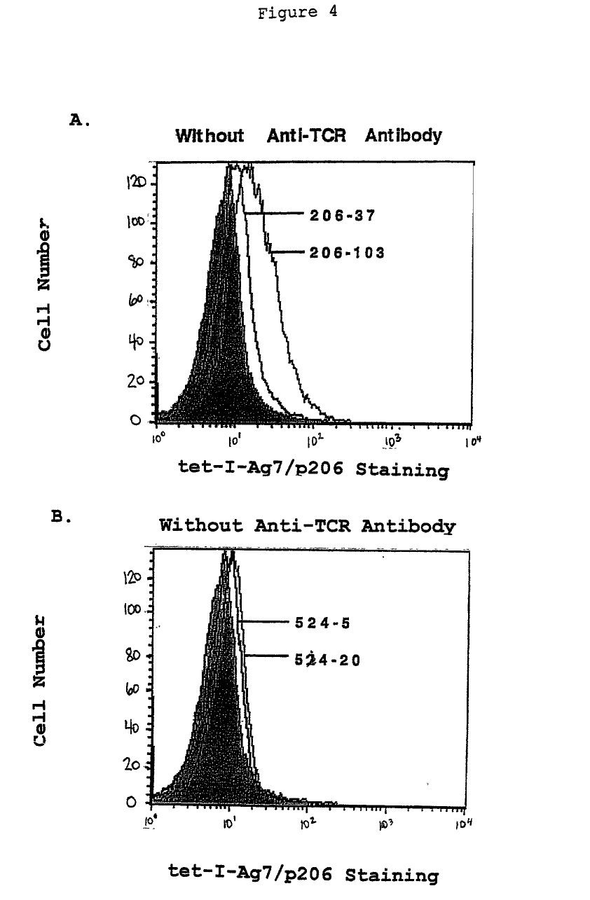

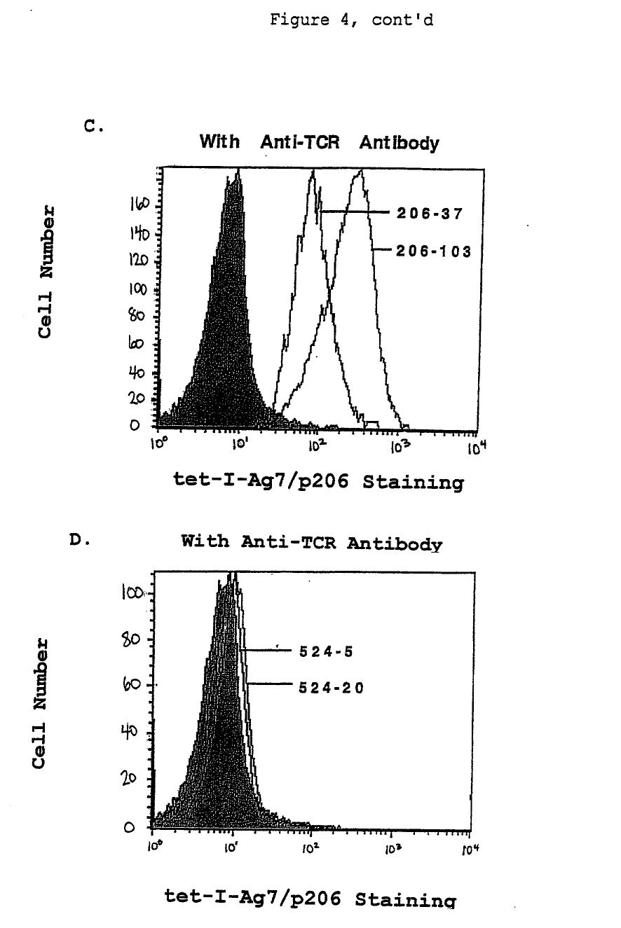

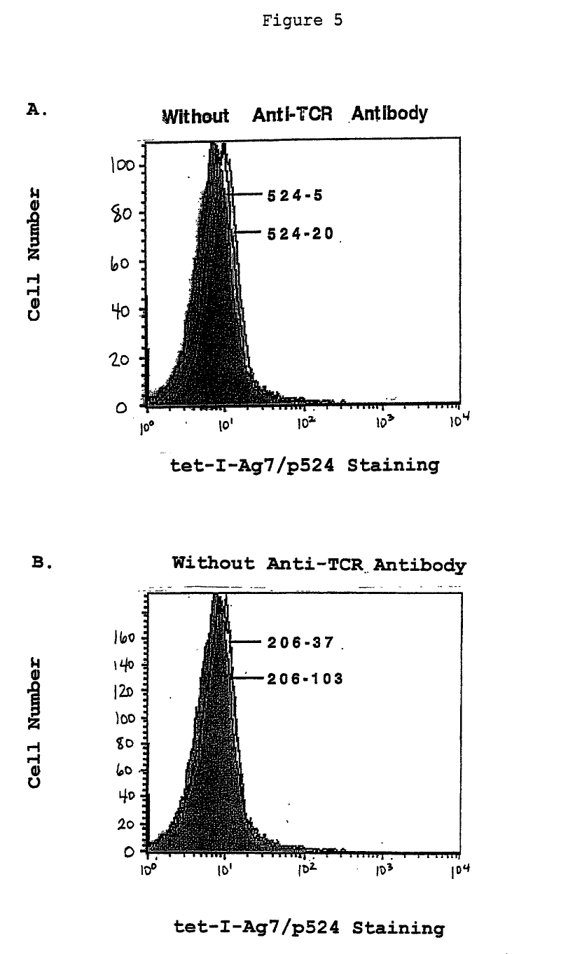

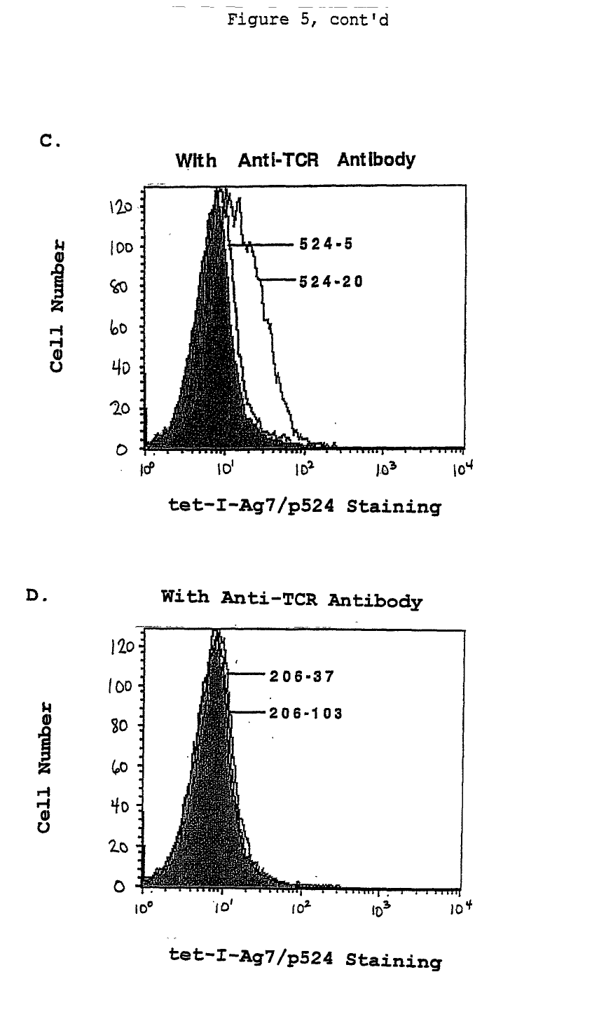

NOD206 and NOD524 hybridoma cells were incubated with each of the two complexes of Example 1 and analyzed using flow cytometry. Surface staining of cells with complexes conjugated with streptavidin-PE was done by incubating the hybridomas at 37° C. for two hours with fluorescent labeled complex in RPMI medium containing 10% FCS, antibiotics, and glutamine. Based on titration studies, the final concentration of complex used in the staining reactions was 25 μg/ml. The results are provided in FIGS. 4 and 5. The tetI-Ag7/p206 complex detected TCRs expressed on NOD206-37 and NOD206-103 cells, but not NOD524-5 or NOD524-20 cells.

Although the staining intensity generally was weak for both hybridoma cells, the NOD206-37 cells stained more weakly than NOD206-103 cells. Since the level of MHC/peptide complex binding to peptide-specific hybridoma cells correlates with the affinity of TCRs for the MHC/peptide ligand, Crawford et al., Immunity 8:675 (1998), the staining results shown in FIG. 4 support the conclusion that NOD206-103 TCRs have a higher affinity for I-Ag7/p206 than NOD206-37 TCRs.

FIG. 5 shows the results of NOD524 cell staining. The tetI-Ag7/p524 complex only weakly stained NOD524-20 TCRs and produced almost no staining of NOD524-5 TCRs. Like that of the tetI-Ag7/p206 reagent, staining of cells using the tetI-Ag7/p524 complex was antigen specific. See FIGS. 4B, 4D, 5B and 5D.

H57 antibody was added to the incubation mixture above at a concentration of 1 μg/10⁶ cells. Addition of H57 antibody to the cells generally increased the intensity of staining by both complexes (compare FIGS. 4A and 5A to FIGS. 4C and 5C). Although staining of NOD524-20 cells by the tetI-Ag7/p524 complex increased significantly in the presence of H57 antibody, staining of TCRs expressed on NOD524-5 cells was not significantly improved. The addition of the H57 antibody enhanced the antigen-specific binding of NOD206 cells more than NOD524 cells. Increased concentration of reagent complexes and incubation time did not further improve the staining significantly.

### Example 7

### Staining of NOD Mouse T Cells Using I-Ag7/p207 Complex

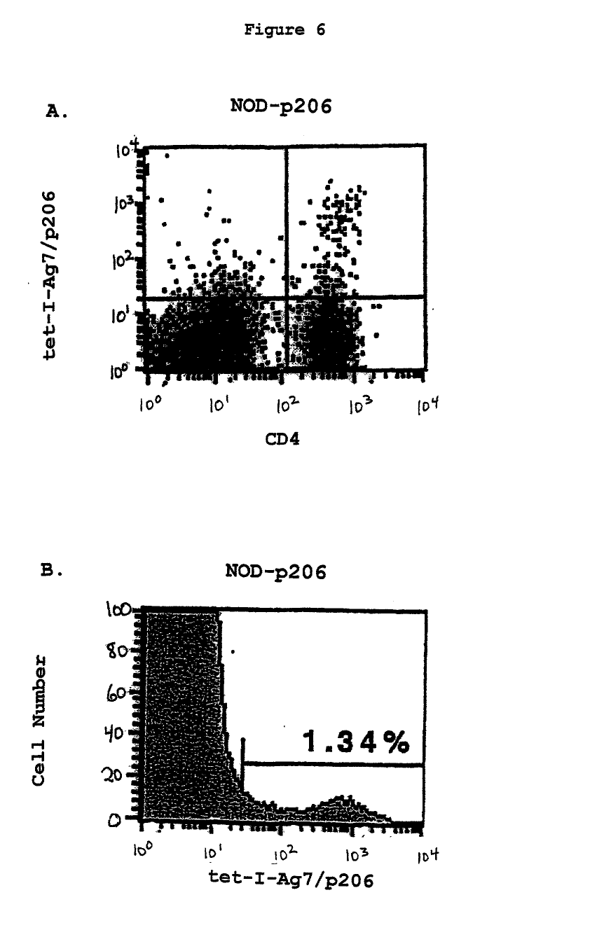

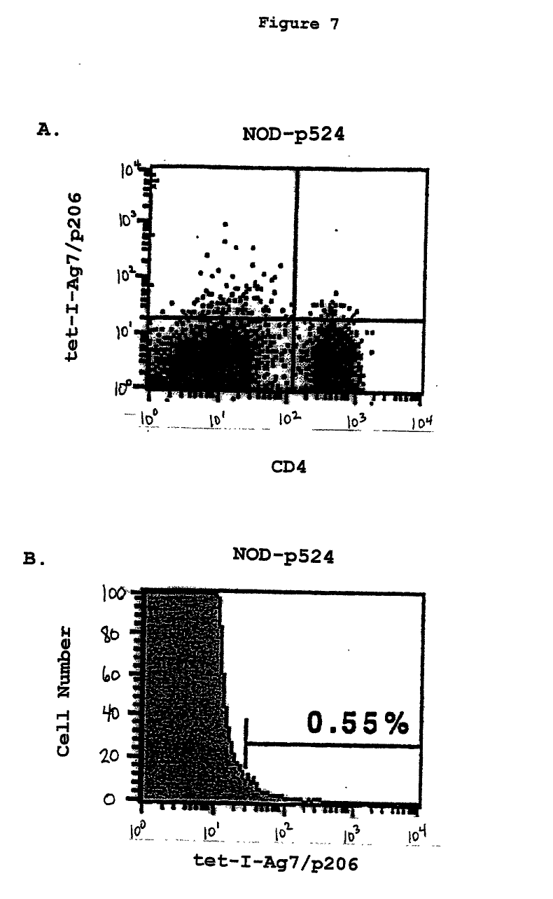

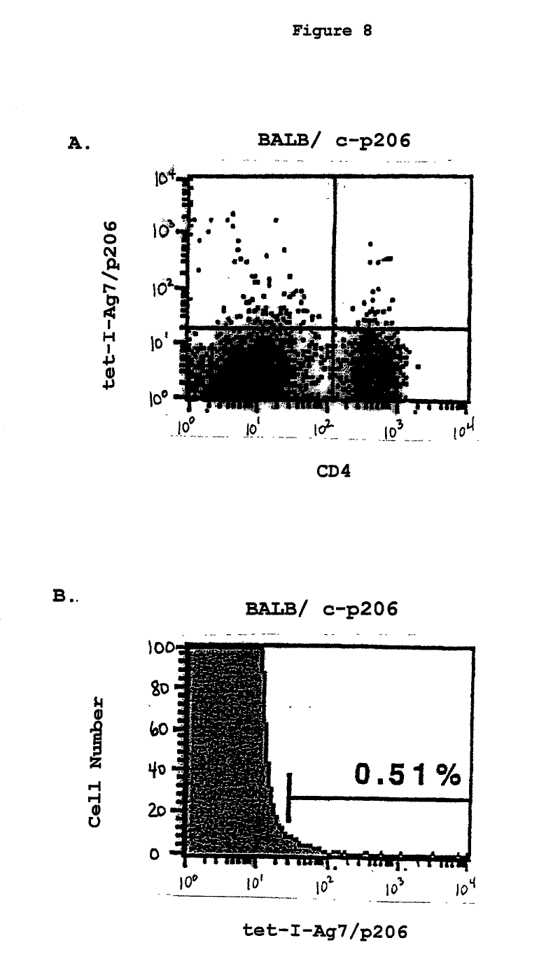

T cells from the draining lymph nodes of immunized 12-week-old NOD mice and from the spleens of non-immunized 12-week-old NOD mice were incubated in RPMI 1640 containing 5% FCS, pH 7.0, at 37° C. with the tetrameric complexes indicated in FIGS. 6-8 at a concentration of 30 μg/ml. After a washing step, cells bearing the fluorescent label were detected by fluorescence activated cell sorting. Cells were also stained for CD4 and CD8 according to known methods. The staining results, given in FIGS. 6-8, show that the tetI-Ag7/p206 complex detected a distinguishable population of CD4+ T cells derived from NOD mice immunized with p206. CD8+ cells were not detected with the complex (data not shown). The population of CD4+ T cells that stained with tetI-Ag7/p206 constituted about 0.6 to 1.0% of the total CD4+ T cell population in the immunized mice, as compared to the total CD4+ T cell population of the negative control BALB/c mice (compare FIGS. 6B and 8B). A similar percentage of CD4+ T cells was detected by the complex in samples from immunized mice of different ages (data not shown). The antigen specificity of the tetI-Ag7/p206 complex was demonstrated. The complex detected a significant population of p206 reactive T cells from mice immunized with p206 but not from mice immunized with p524 (compare FIGS. 6B and 7B).

On the other hand, the same tetI-Ag7/p206 complex did not detect a similar population of T cells in nonimmunized NOD mice (0.03 to 0.05% of these cells were very weakly but consistently detected by the complex; data not shown). This result is consistent with the idea that immunization enhances the selection and/or expansion of T cells expressing TCR which can be detected by the tetI-Ag7/p206 tetramer.

### Example 8

### Staining of NOD Mouse T Cells Using I-Ag7/p524 Complex

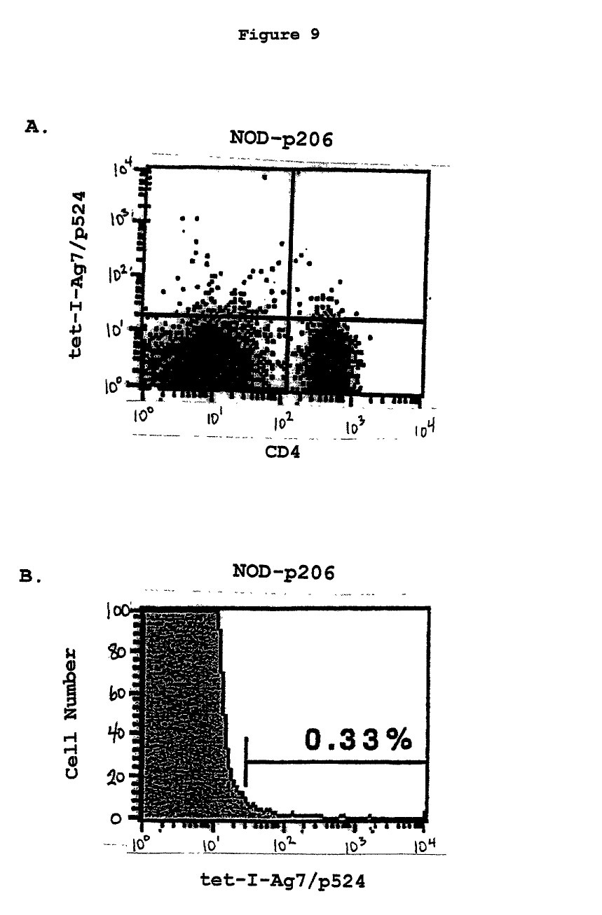

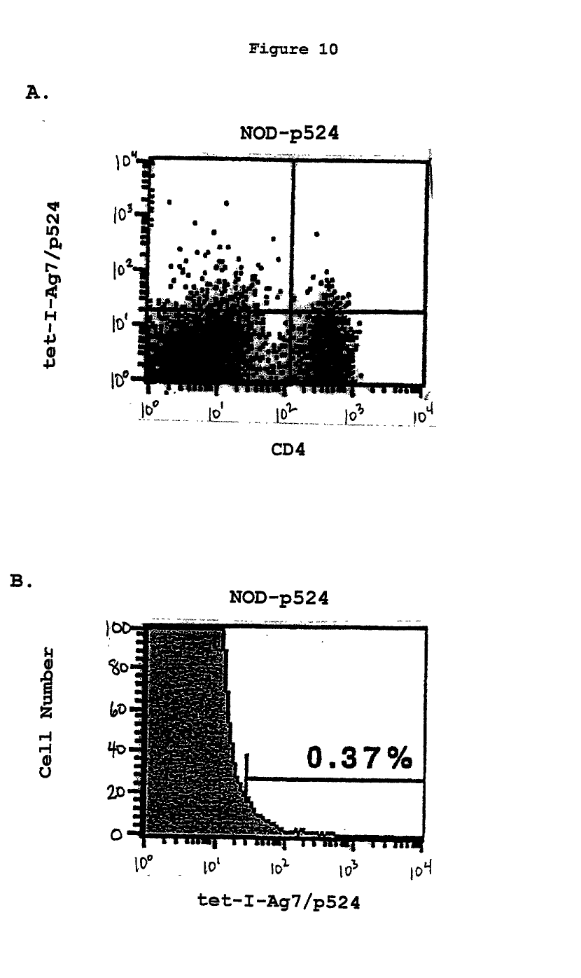

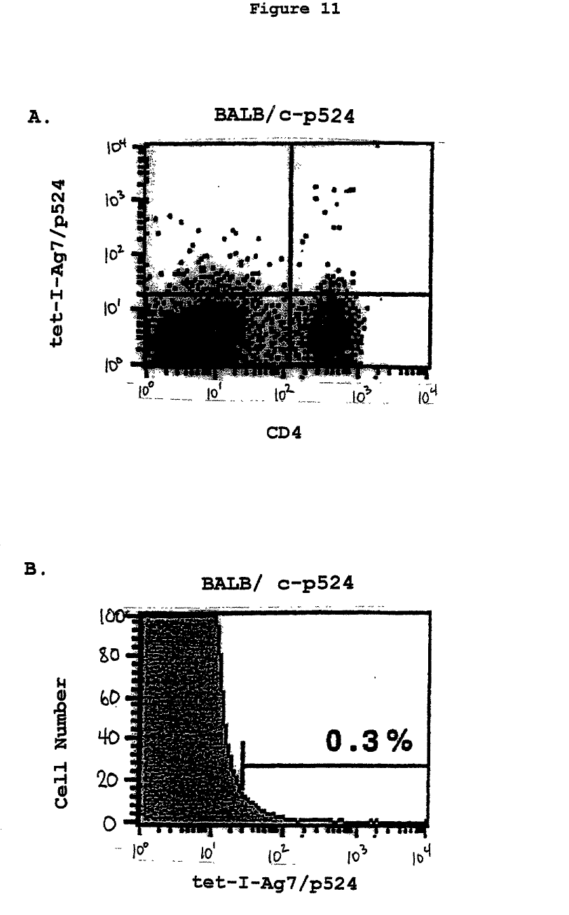

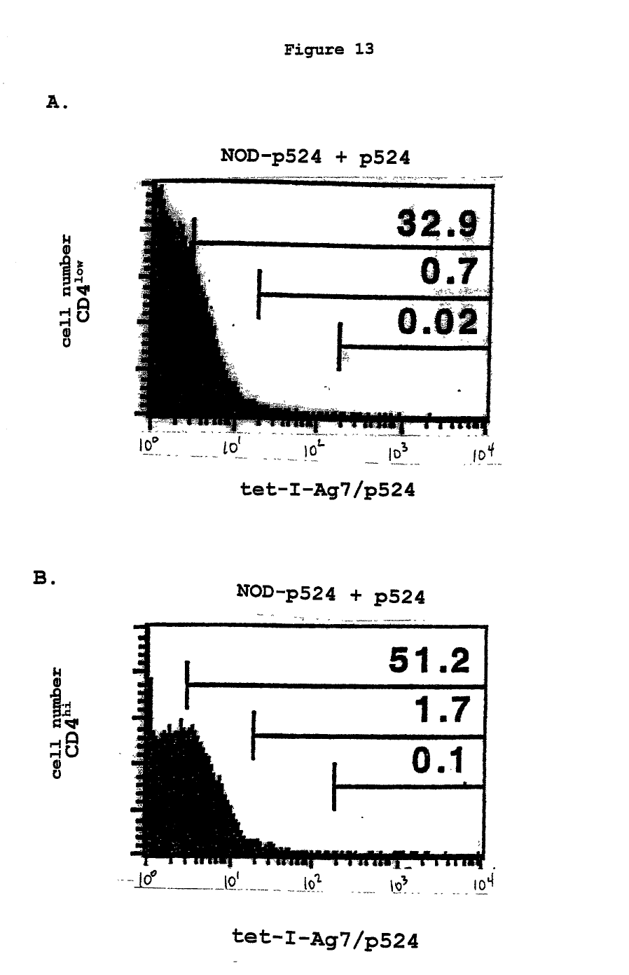

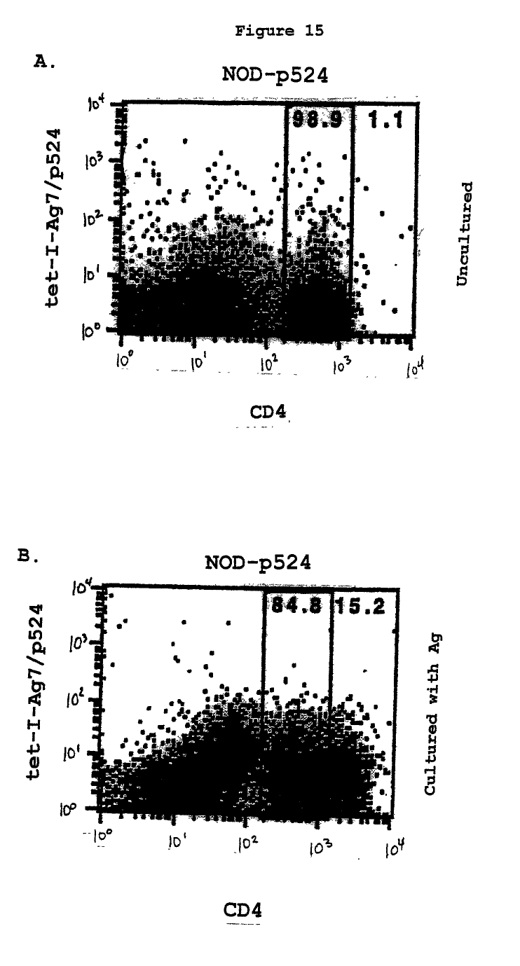

NOD T cells were stained using the methods described in Example 7, with the exception that tetI-Ag7/p524 was substituted for tetI-Ag7/p206. Results are given in FIGS. 9-11. Although the tetI-Ag7/p206 complex identified a distinct population of CD4+ T cells, tetI-Ag7/p524 failed to do so with T cells from NOD mice immunized with p524 (FIG. 10A). Further in vitro culture of the T cells increased the frequency of antigen specific T cells, allowing their detection. See FIGS. 13A, 13B, 15A and 15B. However, a small percentage (0.03 to 0.05%) of CD4+ cells from immunized and nonimmunized NOD mice were consistently stained with the complex (FIGS. 10B and 11B). This result further supports the conclusion that the p524 reactive TCRs have extremely low affinity for tetI-Ag7/p524. Nevertheless, the method disclosed here was able to detect this small population of cells. Immunization and further in vitro stimulation with the antigen was able to enhance the selection and/or expansion of a larger population of T cells expressing p524-reactive TCRs with an affinity high enough to be detected with the tetramer.

### Example 9

### Improved Detection of Antigen Specific T Cells

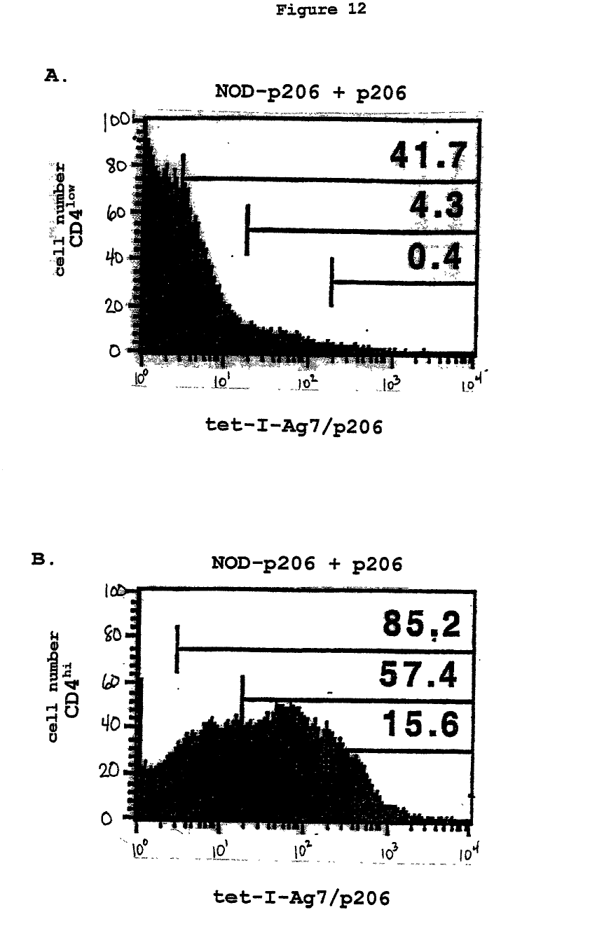

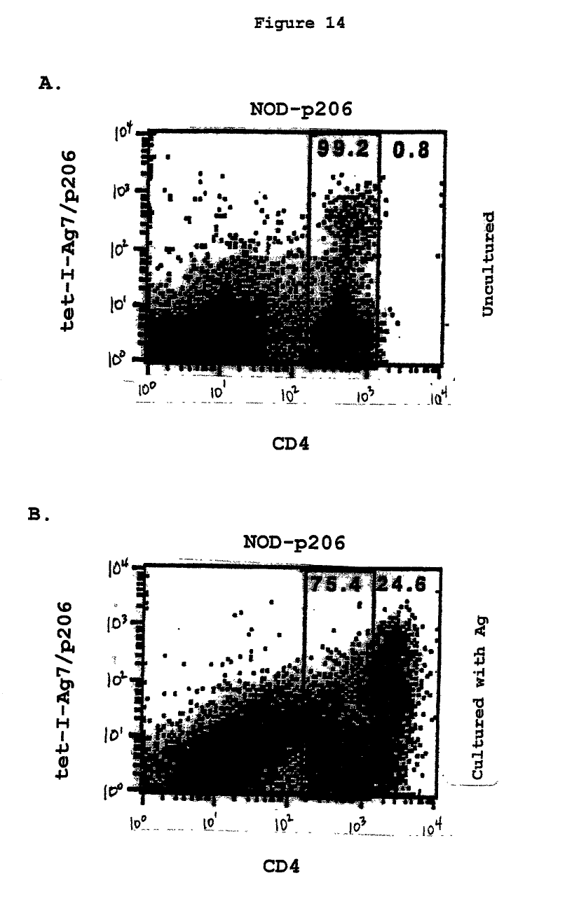

T cells from immunized mice were cultured and restimulated with the antigenic peptide in vitro for 3-4 days in RPMI medium at 37° C. before analysis. After culturing with p206 or p524, a significant portion of CD4⁺ cells upregulated cell surface CD4 expression and became CD4^{hi} cells. See FIGS. 14-15. About 25% of CD4⁺ T cells from p206 immunized mice responded to culturing with p206 by upregulating their CD4 expression, becoming CD4^{hi} cells (FIG. 14). Incubation of T cells from p524 immunized mice with the corresponding peptide antigen in vitro resulted in the production of T cell blasts (data not shown). In addition, a significant portion (15%) of CD4+ T cells increased their CD4 expression and became CD4^{hi} T cells (FIG. 15). Staining with I-Ag7 complex showed that the percentage of CD4⁺ T cells detected by the p206 complex also increased significantly. See FIGS. 12 and 13. When compared to CD4^{low} cells, more than half of the CD4^{hi} cells (about 57%) were detected by the tet-I-Ag7/p206 complex with intermediate to high staining intensity (FIG. 13). Surprisingly, a significant portion of CD4^{hi} T cells were detected either weakly (28%) or not at all (15%). The same tet-I-Ag7/p206 complex did not detect a similarly stained population of lymph node and splenic T cells derived from nonimmunized NOD mice even in the presence of H57 antibody (data not shown). This suggests that a population of p206 reactive T cells may be present spontaneously in the lymph nodes and spleens of NOD mice at a very low frequency, or the affinity of their TCRs is extremely low. The CD4^{hi} T cells were stained very weakly by tet-I-Ag7/p524, however, the average staining intensity of total CD4^{hi} T cells (with a mean channel valve of around 5-6) was 2-3 times higher than that of CD4^{low} T cells (with a mean channel valve of around 2-3). In addition, more CD4^{hi} T cells (1.7%) than CD4^{low} T cells (0.7%) were detected by the complex with intermediate to high staining intensities. See FIG. 13. These results suggest that further activation of p524 reactive CD4⁺ T cells in vitro also can upregulate their CD4 expression and these CD4^{hi} T cells may be weakly detected by the tetramer. Therefore, the complex may bind weakly to the TCRs of almost all p524 reactive T cells in NOD mice.

### Example 10

### Specific Recognition by Antigen-Specific Complexes

As discussed above in detail, soluble recombinant MHC Class II molecules were prepared by infecting insect cells (SF9 and Hi5 cells; PharMingen, San Diego, Calif.) with baculoviruses according to the manufacturer's instructions. DNA constructs were generated by cloning a DNA fragment encoding the GAD peptide, p206, to the 5′ end of a I-Ag7 β chain which lacked the transmembrane and intracellular regions, using a dual-promoter vector pAcUW51. The 3′ end of the I-Ag7 β chain also contained sequences encoding a factor Xa recognition site, and oligohistidine sequence and a peptide which can be biotinylated by the E. coli enzyme BirA. Soluble I-Ag7/p206 complexes were purified using a Ni bead affinity column (Qiagen, Valencia, Calif.) and a Superdex 200 h 10/30 column (Amersham Pharmacia). The purified I-Ag7/p206 complexes were biotinylated using a BirA kit according to the manufacturer's instructions (Avidity, Denver, Co.) and then incubated with streptavidin-phycoerythrin (PharMingen). The multivalent, labeled complexes were purified using a Superdex-200 column.

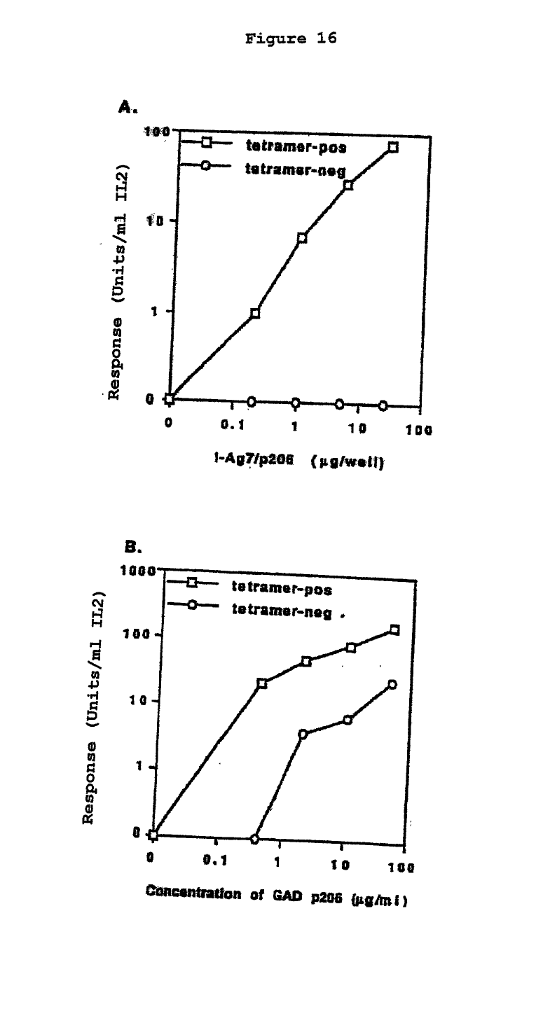

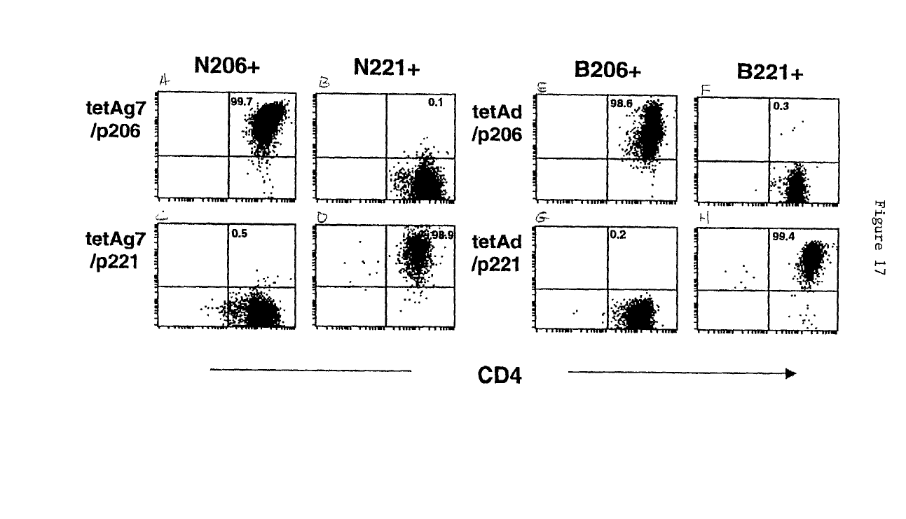

CD4⁺ T cells from mice immunized with p206 were restimulated with the peptide in vitro and incubated for 2-3 hours with the purified, labeled complexes in RPMI containing 10% FCS and blocking reagents at 37° C. The staining reagent was present at 30 μg/ml. Unconjugated H57 antibody (1 μg/10⁶ cells) was included to enhance staining. Cells labeled by the complexes were separated from those which were not recognized by the complexes by FACS. These two groups of cells were tested for reactivity to synthetic p206 peptide and to plate-bound recombinant I-Ag7/p206 protein at different concentrations. See the results in FIG. 16.

The results showed that only CD4⁺ T cells detected and bound by the inventive p206 complex were reactive to the recombinant I-Ag7/p206 protein, while cells which did not bind the complex were not reactive. This suggested that all of the antigen-specific T cells were detected, since no T cells recognizing the antigen recombinant protein were present in the non-detected cells. On the other hand, detected and non-detected cells responded to the synthetic peptide. The reasons for the different response to synthetic peptide and to recombinant protein was unclear, but perhaps was due to contaminants present in the peptide preparation which caused the reaction of contaminant specific T cells.

### Example 11

### Therapeutic Uses of Antigen-Specific Complexes

A GAD peptide specific DQ complex is constructed according to Example 1, using the α and β chains of DQ in place of those of I-Ag7 and using the GAD peptides listed in Table I or immunologically equivalent variants or fragments thereof. The recombinant protein is treated with Factor Xa to remove the His tag and the biotinylation site. A patient suffering from diabetes or a pre-diabetic state is administered the complex, formulated in a pharmaceutical carrier such as normal saline or any other suitable carrier or excipient, depending on the route of administration as discussed above in the specification.

The antigen-specific recombinant complex is administered to patients intraperitoneally, intravenously, orally, intranasally or by any convenient route of administration. Initial doses between 20 to 50 μg/kg are suitable, however those of skill in the art consider it routine to adjust treatment doses to achieve an optimum result with any particular patient. Factors which may be taken into consideration include the affinity and avidity of the binding of the complex, the total and relative numbers of T cells to be treated, the severity of the disease and overall condition of the patient, as well as the judgment of the treating physician. Therefore, the dosage range may be expanded to 10-100 μg/kg, 10-1000 μg/kg, or even higher.

In general, patients are treated with the complex once or twice a week for at least two weeks. One to two weeks after the last treatment, peripheral blood is tested for the presence and function of T cells specific for the antigenic peptide present in the administered recombinant complex. The presence of T cells specific for the antigen are determined by using the antigen specific tetramers. Assays also should include a determination of the proliferation and cytokine secretion response of T cells to the antigenic peptide.

### Example 12

### Isolation and Study of CD4⁺ T Cells from Antigen-Treated Mice

To determine how antigen-based immunotherapy could inhibit IDDM, CD4+, tetramer+ T cells from GAD p206 or p221 immunized NOD mice (N206+ and N221+ cells) were isolated and compared with T cells from similarly immunized BALB/c mice (B206+ or B221+ cells). GAD65 p206 (TYEIAPVFVLLEYVT; SEQ ID NO:1) and p221 (LKKMREIIGWPGGSG; SEQ ID NO:3) were synthesized and then purified by reverse-phase HPLC to a purity of 99.9%. Animals were injected intraperitoneally with 200 μg GAD65 p206 or p221, emulsified in 0.1 ml incomplete Freund's adjuvant (IFA; Sigma, St. Louis, Mo.) on day 0 and day 7. Spleens were removed from animals on day 14. GAD peptide specific CD4+ T cells were isolated using I-Ag7 (tetAg7/p206 or tetAg7/p221) and I-Ad (tetAd/p206 or tetAd/p221) tetramers bound to GAD p206 or GAD p221 produced according to methods known in the art. See Altman et al., Science 274:94-96, 1996; Crawford et al., Immunity 8:675-682, 1998; Liu et al., Proc. Natl. Acad. Sci. USA 97:14596-14601, 2000. The C-terminal end of the α and β chain of I-Ad/p206 heterodimer contained a leucine zipper for heterodimer pairing. See Scott et al., J. Exp. Med. 183:2087-2095, 1996. Splenocytes were isolated from immunized animals and cultured in Click's medium (Life Technologies, Grand Island, N.Y.) plus peptide for three days. Live cells were further maintained in RPMI medium supplemented with IL-2 and restimulated with the indicated antigen once every two to three weeks before analysis. CD4⁺, tetramer⁺ T cells were isolated using FACS and magnetic beads (Milenyi Biotech, Auburn, Calif.). Staining of cells using tetramers was performed according to methods known in the art. Briefly, T cells were stained with PE labeled tetramers plus H57 at 37° C. for 2 to 3 hours and analyzed by FACS using a FACScaliber instrument (Becton-Dickinson, San Jose, Calif.).

Cultured splenocytes from either NOD mice FIGS. 17A-D or BALB/c mice FIGS. 17E-H which had been immunized with the indicated peptides were isolated with the corresponding tetramer and stained with anti-CD4 and labeled tetramers as indicated. The results shown in FIG. 17 were typical of at least four different experiments. Consistent with previous studies, NOD mice immunized with IFA alone, unlike those immunized with CFA, could still develop diabetes (data not shown). Therefore, IFA treatment did not itself alter the function of T cells critical to diabetes development. The I-Ag7 and I-Ad tetramers were shown to be antigen specific as they did not detect a significant number of T cells for mice immunized with the other peptide.

To determine whether the detected tetramer+ T cells were antigen specific, their IL-2 secretion response to synthetic and recombinant GAD peptides (recombinant Ag7/GAD or AD/GAD heterodimers) were measured by ELISA.

Tetramer+ or tetramer− splenocytes were incubated with antigen presenting cells plus either soluble synthetic peptides or plate-bound recombinant peptides covalently linked to I-Ag7 or I-Ad heterodimers for 24 hours. Antigens were 5-fold serially diluted from 25 μg/ml (synthetic peptides) or 25 μg/well (recombinant peptides). HT2 cells were used as the indicator in a MTT assay. Capture ELISA using an assay kit from PharMingen (San Diego, Calif.) was used to measure the amount of cytokines according to the manufacturer's instructions.

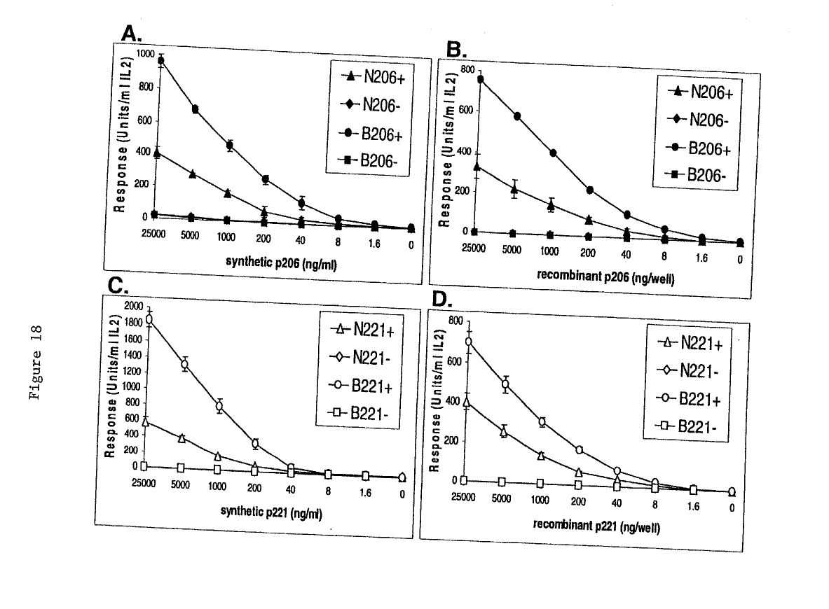

The results were an average of four independent experiments (except for B206+ cells which was from two experiments). The results showed that only tetramer+ but not tetramer− T cells (N206−, N221−, B206− and B221− cells) derived from NOD or BALB/c mice responded to both synthetic (FIGS. 18A and 18C) and recombinant (FIGS. 18B and 18D) peptides by secreting IL-2. These results suggested that the response was not unique to peptides made by chemical synthesis which may lead to altered peptide display by Class II MHC. Therefore, the response was antigen dependent and the cells did not respond to irrelevant peptides. In addition, tetramer+ T cells specific for either p206 or p221 from BALB/c mice secreted 2- to 3-fold more IL-2 than did T cells from NOD mice.

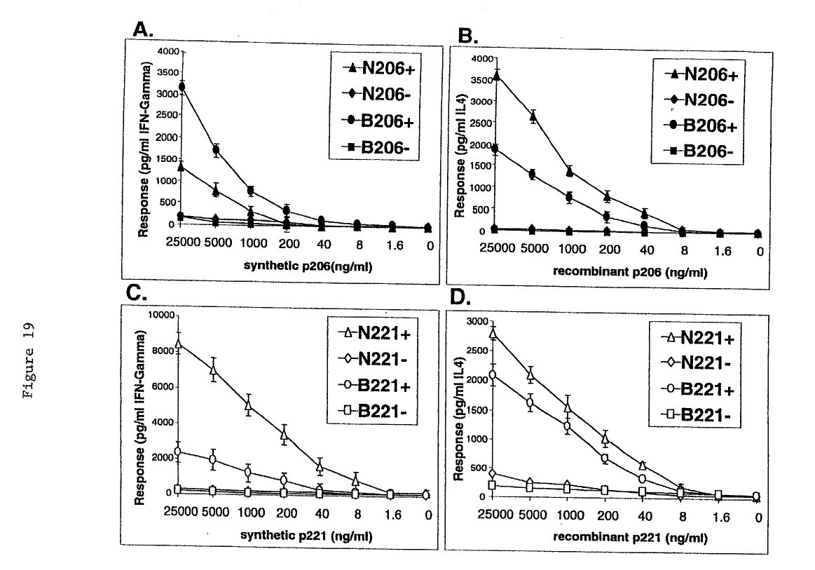

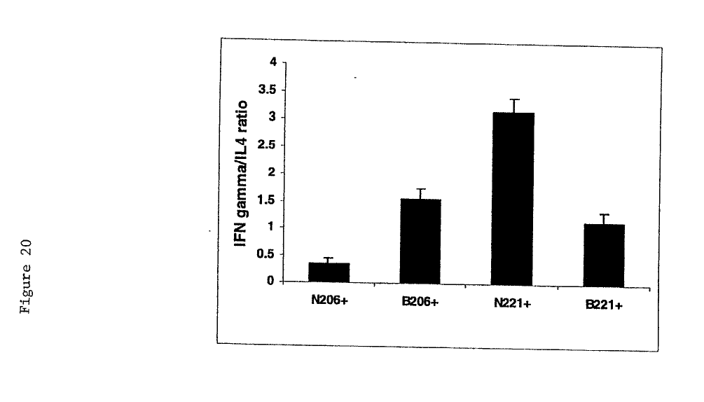

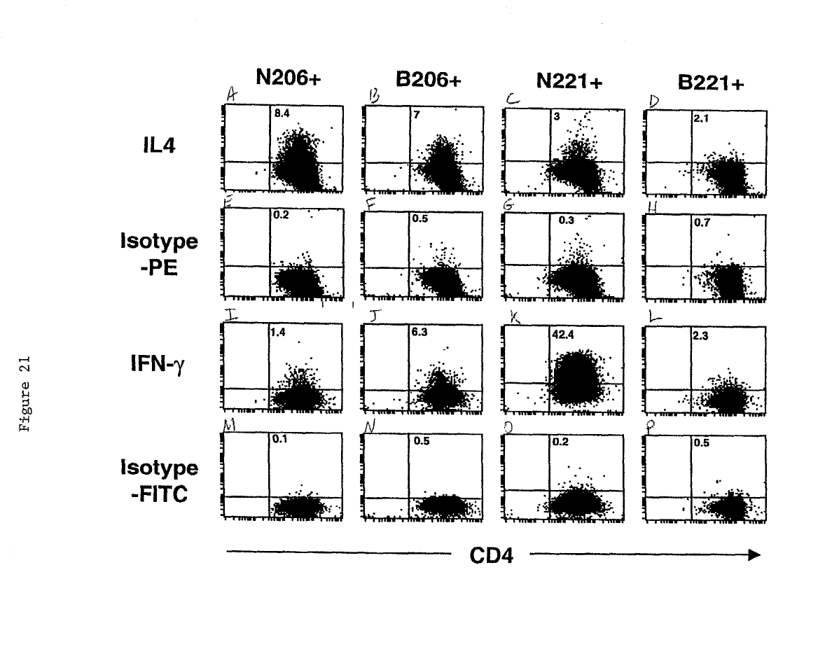

The cytokine secretion profiles of N206+ and N211+ cells were determined and compared with the profiles of B206+ and B221+ cells following antigen stimulation. Cells were stimulated with various concentrations of antigens as described above. Cell culture supernatant was harvested after 24 hours for ELISA. ELISA analyses showed that only tetramer+ T cells secreted detectable amount of IFNγ and IL-4. See FIG. 19. Specifically, N206+ cells secreted about 2- to 3-fold less IFNγ but 2-fold more IL-4 than did B206+ cells. See FIGS. 19A and 19B. In contrast, N221+ cells secreted approximately 4-fold more IFNγ and 1.5-fold more IL-4 than did B221+ cells. See FIGS. 19C and 19D. Compared to N206+ cells, N221+ cells secreted between 8- to 10-fold more IFNγ and comparable amounts of IL-4. The IFNγ/IL-4 ratio of the amount of cytokines that were secreted at the antigen (synthetic peptide) concentration of 25 μg/ml is shown in FIG. 20. The results shown are an average of at least four independent experiments. The lower detection limit was 8 pg/ml for IL-4 and 30 pg/ml for IFNγ. The IFNγ/IL-4 ratio for N206+ cells were 4-fold less than that for B206+ cells (0.38 vs. 1.6) and 8-fold less for N221+ cells (0.38 vs. 3.1). See FIG. 20. The ratio for B206+ cells was similar to that for B221+ cells (1.6 vs. 1.3).

Intracellular cytokine staining (ICS) of tetramer+ cells were also performed. The cells were stimulated with PMA and ionomycin in the presence of brefeldin A for 6 hours at 37° C. Cells were then stained for cell surface antigen (CD4), fixed with paraformaldehyde, permeabilized, and then intracellularly stained with anti-IFNγ or anti-IL-4 antibodies according to known methods. Briefly, T cells were resuspended in 0.1% saponin buffer (w/v; Sigma, St. Louis, MO). The cells then were stained intracellularly using antibodies against cytokines, or by negative isotype control antibodies (PharMingen). Isotype controls are shown right below the cytokine antibody staining. The results shown in FIG. 21, are the representative of four independent experiments.

Although not all cells were stimulated to produce the cytokines, the relative percentage of cells producing each cytokine correlates with the ELISA results except for B206+ cells producing IL-4. See FIG. 21. Taken together, the results suggest that N206+ cells are biased toward Th2 subset while N221+ cells are more like Th1 cells. Therefore, treatments of NOD mice with the two GAD peptides induce different Th cell subsets. On the other hand, the cells from BALB/c are essentially nonpolarized, although they are slightly biased toward Th1 cells. Additionally, ICS studies also showed that a small population of these tetramer+ cells (0.3-0.5%) might co-produce IL-4/IFNγ suggesting that they are Th0 cells (data not shown).

### Example 13

### Therapeutic Transfer of Peptide-Responsive T Cells Into Non-Diabetic NOD/Scid Mice Inhibits Diabetes Development

To study the role of NOD mouse T cells in diabetes, a population of peptide specific T cells (either N206+ or N221+ cells, detected by tetramer reagents) were co-transferred together with non-diabetic NOD mouse splenocytes into NOD/scid mice. Co-transfer of B206+ and B221+ cells with NOD mouse splenocytes were not performed due to the expected allogeneic responses to NOD splenocytes. Seven week old NOD/scid mice received a single i.v. injection of NOD mouse splenocytes (10×10⁶ cells/mouse) co-transferred with either N206+ or N221+ T cells (double transfer) (5×10⁶ cells/mouse), or with both N206+ (2.5×10⁶ cells/mouse) and N221+ T cells (2.5×10⁶ cells/mouse)(triple transfer). Eleven to 12 animals were used in each transfer experiment. Recipient mice were monitored for up to 30 weeks of age and were considered diabetic after two consecutive weeks of glycosurea >2% and blood glucose level >250 μg/ml.

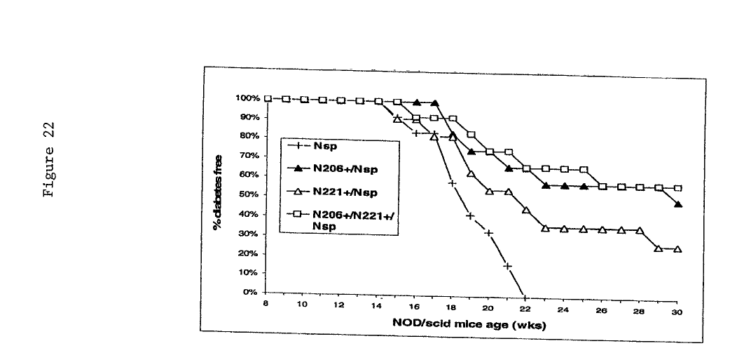

Both N206+ and N221+ cells inhibited the development of diabetes precipitated by the transferred NOD mouse splenocytes. See FIG. 22. Mice that received N206+ cells showed a slightly delayed onset of diabetes, and 50% of the animals remained diabetes-free compared to mice that received NOD splenocytes alone at 30 weeks of age. In comparison, the time of onset of diabetes in mice that received N221+ cells did not change but 27% of the animals remained diabetes free. Therefore, N206+ cells demonstrated a stronger inhibitory effect than did N221+ cells on diabetes development. These differences are probably not due to the number of tetramer+ T cells present in recipient animals as a comparable number of N206+ cells and N221+ cells together with NOD mouse splenocytes did not show a synergistic effect on diabetes development. The results were essentially identical to those of the double-transferred mice receiving N206+ cells. In addition, non-diabetic animals that received T cells did not develop detectable insulitis (data not shown). NOD/scid mice receiving peptide-specific T cells in the absence of NOD mouse splenocytes did not become diabetic and were not detectable in the spleens of recipient mice (data not shown), probably because other types of cells, such as CD8+ T cells or B cells, which are absent in NOD/scid mice, are required for the transferred peptide-specific T cells to expand and function.

### Example 14

### IL-10 and TGFβ Secretion Profiles of Peptide-Specific T Cells

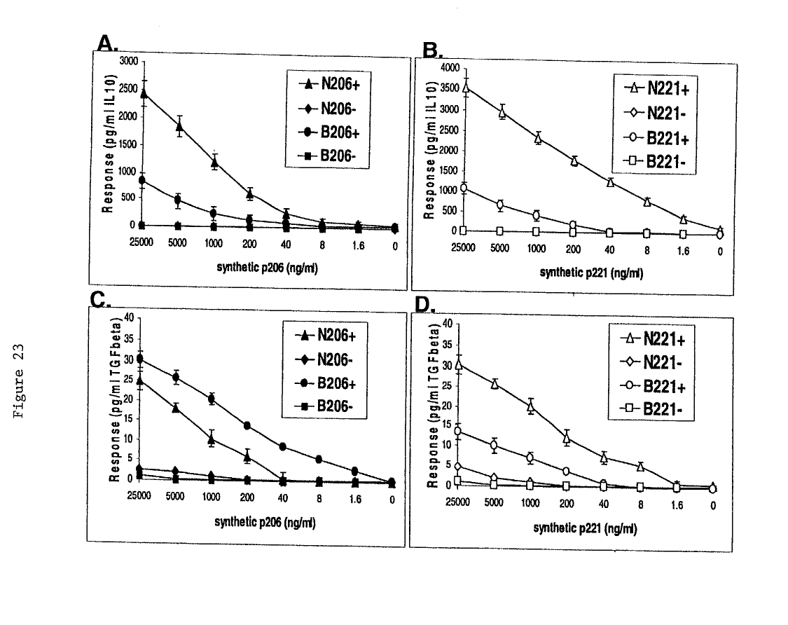

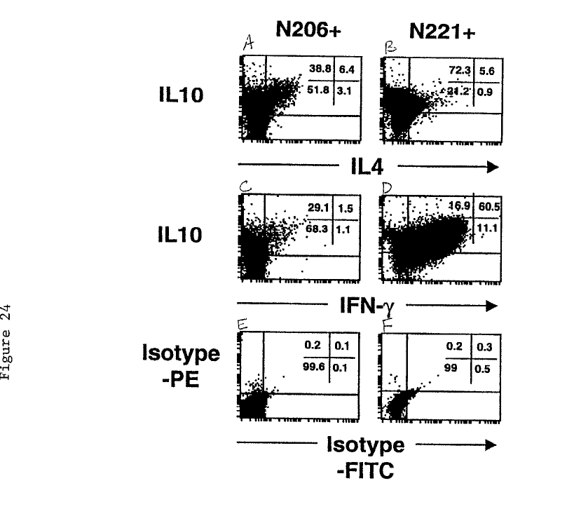

One explanation for the inhibitory effect of tetramer+ T cells on diabetes development was that these cells secreted IL-10 or TGFβ which regulated the autoimmune response. Shevach, Annu. Rev. Immunol. 18:423-449, 2000; Sakaguchi, Curr. Opin. Immunol. 12:684-690, 2000; Roncarlo and Levings, Curr. Opin. Immunol. 12:676-683, 2000; Maloy and Powrie, Natl. Immunol. 2:816-822, 2001; Moore et al., Annu. Rev. Immunol. 19:683-765, 2001. To examine this possibility, the IL-10 and TGFβ secretion profiles of antigen-stimulated tetramer+ T cells were determined. ELISA results showed that N221+ cells secreted about 1.5-fold more IL-10 than did N206+ cells. See FIGS. 23A and 23B. In contrast to the IL-2 results, both N206+ and N221+ cells secreted approximately 3- to 4-fold more IL-10 than did the B206+ and B221+ cells. See FIGS. 23A and 23B. All tetramer+ T cells secreted very little TGFβ. See FIGS. 23C and 23D. The lower detection limit for IL-10 and TGFβ in FIG. 23 was 30 pg/ml and 15 pg/ml, respectively. The results shown are an average of four independent experiments.

N206+ and N221+ cells were also co-stained intracellularly with anti-IFNγ antibodies. Essentially all IL-4 producing N206+ or N221+ cells co-produced IL-10, suggesting that they are Th2 cells. See FIG. 24. Interestingly, all IFNγ-producing N206+ cells also produced IL-10, suggesting that they are not classical Th1 cells but more likely to be T regulatory type 1 (Tr1) cells as described by Groux et al., Nature 389:737-742, 1997. On the other hand, although the majority of IFNγ-producing N221+ cells also co-produced IL-10, a small proportion of the cells did not. The former cells therefore were likely Tr1 cells and the latter cells Th1 cells. The population of N206+ cells contained 4-fold more Th2 cells and 25-fold fewer Tr1 cells than did N221+ cells. Therefore, the N206+ and N221+ cells also differed from each other in that they contained various Th and Tr cell populations that produce different combinations of cytokines.

1. A recombinant nucleic acid which comprises DNA encoding an antigenic peptidic sequence which binds to a Class II MHC molecule and DNA encoding the extracellular portion of the β chain of said Class II MHC molecule.

2. A recombinant nucleic acid according to claim 1 which further comprises DNA encoding the extracellular portion of the α chain of said Class II MHC molecule.

3. A recombinant nucleic acid according to claim 1, wherein said Class II MHC β chain lacks a complete transmembrane region.

4. A recombinant nucleic acid according to claim 2, wherein said Class II MHC β chain and said Class II MHC α chain lack complete transmembrane regions.

5. A recombinant nucleic acid according to claim 1, wherein said peptidic sequence which specifically binds to a Class II MHC molecule is an autoantigen.

6. A recombinant nucleic acid according to claim 5, wherein said autoantigen is a multiple sclerosis autoantigen.

7. A recombinant nucleic acid according to claim 5, wherein said autoantigen is an experimental autoimmune encephalomyelitis autoantigen.

8. A recombinant nucleic acid according to claim 5, wherein said autoantigen is a diabetic autoantigen.

9. A recombinant nucleic acid of claim 8, wherein said diabetic autoantigen is a fragment of glutamic acid decarboxylase.

10. A recombinant nucleic acid of claim 9, wherein said fragment of glutamic acid decarboxylase comprises a sequence selected from SEQ ID NOS: 1-13 or immunologically equivalent variants or fragments thereof.

11. A recombinant nucleic acid of claim 1, wherein said DNA encoding a peptidic sequence which specifically binds to said Class II MHC molecule encodes SEQ ID NO: 1.

12. A recombinant nucleic acid of claim 1, wherein said DNA encoding a peptidic sequence which specifically binds to said Class II MHC molecule encodes SEQ ID NO: 2.

13. A recombinant nucleic acid of claim 1 which further comprises DNA encoding a biotinylation site.

14. A recombinant nucleic acid of claim 1 which further comprises DNA encoding an oligohistidine sequence.

15. A recombinant nucleic acid of claim 2 which further comprises DNA encoding a biotinylation site.

16. A recombinant nucleic acid of claim 2 which further comprises DNA encoding an oligohistidine sequence.

17. A recombinant protein which is encoded by the recombinant nucleic acid of claim 1.

18. A recombinant protein which is encoded by the recombinant nucleic acid of claim 2.

19. A recombinant protein which is encoded by the recombinant nucleic acid of claim 9.

20. A recombinant protein which is encoded by the recombinant nucleic acid of claim 10.

21. A recombinant protein which is encoded by the recombinant nucleic acid of claim 11.

22. A recombinant protein which is encoded by the recombinant nucleic acid of claim 12.

23. A recombinant protein which comprises a preselected peptidic antigen which binds to a Class II MHC molecule, the extracellular portion of a β chain of said Class II MHC molecule, and the extracellular portion of an α chain of said Class II MHC molecule.

24. A recombinant protein according to claim 23 which further comprises a biotinylation site.

25. A recombinant protein according to claim 23 which further comprises an oligohistidine sequence.

26. A recombinant protein according to claim 23 wherein said peptidic sequence is an autoantigen.

27. A stable molecular complex which comprises a recombinant protein according to claim 17.

28. A stable molecular complex which comprises a recombinant protein according to claim 18.

29. A stable molecular complex which comprises a recombinant protein according to claim 23.

30. A stable molecular complex which comprises a recombinant protein according to claim 24.

31. A stable molecular complex which comprises a recombinant protein according to claim 25.

32. A stable molecular complex according to claim 30 which further comprises a biotin covalently linked to said recombinant protein.

33. A stable molecular complex according to claim 30 which further comprises an effector-avidin bound to said biotin.

34. A stable molecular complex according to claim 33, wherein said effector is selected from a label and a toxin.

35. A stable molecular complex according to claim 23, wherein said peptidic antigen is a diabetic autoantigen.

36. A method of detecting T cells which recognize a preselected peptidic antigen in a population of T cells which comprises:
(a) providing a stable molecular complex according to claim 28, wherein said peptidic sequence is said preselected peptidic antigen and wherein said stable molecular complex is labeled;
(b) incubating said stable molecular complex with said population of T cells under conditions such that said stable molecular complex binds to T cells in said population of T cells which recognize said preselected peptidic antigen;
(c) optionally removing unbound complexes; and
(d) detecting said labeled complexes on said T cells which recognize said preselected peptidic antigen.

37. A method of detecting T cells which recognize a preselected peptidic antigen in a population of T cells according to claim 36 which further comprises, between steps (a) and (b), stimulating said population of T cells by contacting said T cells with said preselected peptide antigen or allogeneic antigen presenting cells which present said preselected peptidic antigen.

38. A method of diagnosing a diabetic or pre-diabetic condition in a mammal which comprises:
(a) obtaining a sample which contains a population of T cells from said mammal;
(b) providing a stable molecular complex according to claim 28, wherein said antigenic peptidic sequence is a diabetic autoantigen;
(c) incubating said stable molecular complex with said sample under conditions such that said stable molecular complex binds to T cells in said sample which recognize said diabetic autoantigen;
(d) optionally removing unbound complexes; and
(e) determining whether said stable molecular complex has bound to any T cells in said sample.

39. A method of diagnosing a diabetic or pre-diabetic condition in a mammal according to claim 38 which further comprises between steps (a) and (b), stimulating said sample of T cells by contacting said T cells with said preselected peptide antigen or allogeneic antigen presenting cells which present said preselected peptidic antigen.

40. A method according to claim 36, wherein said conditions of incubation include addition of anti T cell receptor antibody.

41. A method according to claim 37, wherein said conditions of incubator include addition of anti T cell receptor antibody.

42. A method according to claim 38, wherein said anti T cell receptor antibody is present at a concentration of about 0. 1 μg per 10⁶ cells to about 10 μg per 10⁶cells.

43. A method according to claim 39, wherein said anti T cell receptor antibody is present at a concentration of about 0.1 μg per 10⁶ cells to about 10 μg per 10⁶ cells.

44. A method of inducing tolerance to a preselected peptidic antigen in a population of T cells which comprises:
(a) providing a stable molecular complex according to claim 28; and
(b) contacting said stable molecular complex with said T cells.

45. A method of inducing or expanding protective clones of T cells which recognize a preselected antigen in a population of T cells which comprises:
(a) providing a stable molecular complex according to claim 28; and
(b) contacting said stable molecular complex with said T cells.

46. A method of killing T cells which recognize a preselected peptidic antigen in a population of T cells which comprises:
(a) providing a stable molecular complex according to claim 34 wherein said effector is a toxin; and
(b) contacting said stable molecular complex with said T cells.

47. A method of vaccinating a patient against a preselected peptidic antigen which comprises:
(a) providing a stable molecular complex according to claim 23; and
(b) administering said stable molecular complex to said patient wherein specific T cell clones recognizing said preselected peptidic antigen are expanded.

48. A method of inhibiting the onset of diabetes in a mammal in need thereof, which comprises:
(a) providing a stable molecular complex according to claim 28, wherein said antigenic peptide sequence is a diabetic autoantigen;
(b) contacting said stable molecular complex with a population of T cells allogeneic to said mammal under conditions such that said stable molecular complex binds to T cells in said population that recognize said diabetic autoantigen;
(c) separating from said T cell population T cells that bind to said stable molecular complex; and
(d) administering said separated T cells to said mammal.
